(19) United States
(12) Patent Application Publication
(10) Pub. No.: US 2026/0272993 A1
(43) Pub. Date: Sep. 17, 2026

(54) Method of Treatment

(71) Applicants: HUDSON INSTITUTE OF MEDICAL RESEARCH, Victoria (AU); MONASH UNIVERSITY, Victoria (AU); MONASH HEALTH, Clayton (AU)

(72) Inventors: Charlotte Yuen Yu Keung, Heidelberg Heights (AU); Gregory Thomas Charles Moore, Kew (AU); Rebecca Seok Wai Lim, Houston, TX (US)

(21) Appl. No.: 19/480,113

(22) PCT Filed: May 3, 2024
(86) PCT No.: PCT/AU2024/050434

§ 371 Date: Oct. 30, 2025

(30) Foreign Application Priority Data
May 4, 2023 (AU) 2023901341
Dec. 1, 2023 (AU) 2023903893

(51) Int. Cl. A61K 35/50; A61P 37/06

(52) U.S. Cl. CPC A61K 35/50; A61P 37/06

(57) ABSTRACT

The present invention relates generally to methods and compositions for treating and/or preventing an inflammatory bowel disease (IBD) and perianal fistulas, the method comprising the administration of therapeutic cells in the subject in need thereof.

### RELATED APPLICATIONS

This application is a National Stage Application under 35 U.S.C. 371 of co-pending PCT application PCT/AU2024/050434 designating the United States and filed May 3, 2024; which claims priority to Australian Provisional Application No. 2023901341 entitled “Method of Treatment” filed 4 May 2023 and Australian provisional patent application No. 2023903893 entitled “Method of Treatment” filed 1 Dec. 2023, the contents of which are incorporated herein by reference in their entirety.

### FIELD

The present invention relates generally to methods and compositions for treating and/or preventing an inflammatory bowel disease (IBD) in a subject.

### BACKGROUND

Fistulas are abnormal connection between two body parts, such as an organ or blood vessel and another structure that are normally not connected. Fistulas often result in abnormal flow of fluid or material from one space to the other and typically arise from an infection, inflammation, injury or surgery. For example, fistulas may form during surgical and medical treatment. Complications or trauma, from surgery or abnormal healing and/or injury during childbirth, can also lead to fistulas.

Fistulas can also form as a result of inflammatory diseases such as IBD, in particular, in the perianal, anal and vaginal regions.

Inflammatory bowel disease (IBD) is a group of complex inflammatory conditions of the colon and small intestine, which includes Crohn's disease (CD) and ulcerative colitis (UC). Crohn's Disease and Ulcerative Colitis are lifelong gastrointestinal disorders that commonly present at a young age and are increasing in incidence and prevalence. In developed countries, the incidence of IBD is 100-220 in 100 000.

Because of its onset being in young adults, IBD has a profound impact on productive employment and social milestones. The pathophysiology of this complex disease includes genetic predisposition and intestinal microbial dysbiosis.

Fibrosis is a common and incurable complication of IBD. When fibrotic complications occur, patients often have to undergo disfiguring surgery. Approximately 50% of patients with CD will develop complicated disease characterized by stricture and or fistula formation within five years of diagnosis. Complex Crohn's perianal fistulas are one of the most difficult to treat conditions in inflammatory bowel disease.

Approximately one third of CD patients will require surgery due to fibrotic complications. Surgery unfortunately does not protect patients from disease recurrence and fibrotic complications that will then require further surgery. Effective regenerative therapies, which include anti-fibrotic and/or anti-inflammatory properties are required to prevent and treat disease progression.

Previous protocols for the treatment of IBD have involved injection of mesenchymal stem cells (MSC). However, these therapies have been limited by cost and the requirement for specialised facilities for tissue and cell retrieval, cell revival and culture, prior to use.

Hence, there remains an urgent need for improved methods of treating fistulas, including fistulas that occur in IBD, in particular Crohn's perianal fistulas.

### SUMMARY

The present disclosure is predicated, at least in part, on the inventors' unexpected finding that human amnion epithelial cells (hAECs) have both short and longer-term therapeutic benefit in the treatment of fistulas, including fistulas associated with IBD, in particular Crohn's Disease and perianal fistulising Crohn's Disease.

Accordingly, in one aspect, there is provided a method for treating a fistula in a subject, the method comprising administering to a subject in need thereof a therapeutically effective amount of human amnion epithelial cells (hAECs), wherein prior to administration, the hAECs are screened and selected for their ability to suppress T cell activity.

In another aspect disclosed herein, there is provided a method for treating an inflammatory bowel disease in a subject, the method comprising administering to a subject in need thereof a therapeutically effective amount of human amnion epithelial cells (hAECs), wherein prior to administration, the hAECs are screened and selected for their ability to suppress T cell activity.

In some embodiments, the inflammatory bowel disease is ulcerative colitis. In some embodiments, the inflammatory bowel disease is Crohn's Disease. In some embodiments, the subject has perianal fistulising Crohn's Disease.

In some embodiments, the hAECs are screened and selected for their ability to suppress T cell proliferation by at least about 25%.

In some embodiments, the subject has a complex perianal fistula. In some embodiments, the subject has one or more of intersphincteric, trans-sphincteric; extra-sphincteric; and supra-sphincteric fistulas. In some embodiments, the subject has a rectovaginal fistula.

In a particular embodiment, the subject has a complex perianal fistula that is refractory to treatment with an immune modulating agent for a period of at least about 3 months. Suitable immune modulating agents will be familiar to persons skilled in the art, an illustrative example of which includes a tumour necrosis factor-α (TNF-α) inhibitor, such as an anti-TNF-α antibody. In an embodiment, the subject has a complex perianal fistula that is refractory to treatment with an anti-tumour necrosis factor-α (TNF-α) antibody for a period of at least about 3 months.

In some embodiments, the hAEC are administered to a fistula of the subject in an amount of from about 1×10⁶ to about 1×10⁹ hAECs per fistula. In some embodiments, the hAEC are administered to a fistula of the subject in an amount of from about 1×10⁶ to about 1×10⁸ hAECs per fistula. In some embodiments, the hAEC are administered to a fistula of the subject in an amount of from about 1×10⁶ to about 1×10⁷ hAECs per fistula. In another embodiment, the hAEC are administered to a fistula of the subject in an amount of from about 10×10⁶ to about 80×10⁶ hAECs per fistula. In another embodiment, the hAEC are administered to a fistula of the subject in an amount of from about 30×10⁶ to about 50×10⁶ hAECs per fistula. In yet another embodiment, the hAEC are administered to a fistula of the subject in an amount of about 40×10⁶ hAECs per fistula. In another embodiment, the hAEC are administered to a fistula of the subject in an amount of about 120×10⁶ hAECs per fistula.

In an embodiment, the subject is a human.

In an embodiment, the hAECs are autologous or allogenic or xenogeneic to the subject. In another embodiment, the hAECs are allogeneic to the subject.

In some embodiments, the hAECs are immortalised hAECs.

In some embodiments, following said screening and selection, the hAECs are cryopreserved and subsequently thawed immediately prior to administration to the subject.

In another embodiment, the subject has perianal fistulising Crohn's Disease and said treatment provides a reduction in active fistula number and/or size at 24 weeks following administration of the hAECs. In yet another embodiment, the subject has perianal fistulising Crohn's Disease and said treatment provides a reduction in active fistula number and/or size at 52 weeks following administration of the hAECs. In yet another embodiment, the subject has perianal fistulising Crohn's Disease and said treatment provides a reduction in active fistula number and/or size at 104 weeks following administration of the hAECs.

In another aspect, there is provided a use of human amnion epithelial cells (hAECs) in the manufacture of a medicament for treating a fistula in a subject, wherein the hAECs formulated for administration to a subject in need thereof, and wherein prior to said administration, the hAECs are screened and selected for their ability to suppress T cell activity.

In another aspect, there is provided a use of human amnion epithelial cells (hAECs) in the manufacture of a medicament for treating inflammatory bowel disease in a subject, wherein the hAECs formulated for administration to a subject in need thereof, and wherein prior to said administration, the hAECs are screened and selected for their ability to suppress T cell activity.

In some embodiments, the inflammatory bowel disease is ulcerative colitis. In some embodiments, the inflammatory bowel disease is Crohn's Disease. In some embodiments, the subject has perianal fistulising Crohn's Disease.

In some embodiments, the hAECs are screened and selected for their ability to suppress T cell proliferation by at least about 25%.

In some embodiments, the subject has a complex perianal fistula. In some embodiments, the subject has one or more of intersphincteric, trans-sphincteric; extra-sphincteric; and supra-sphincteric fistulas. In some embodiments, the subject has a rectovaginal fistula.

In a particular embodiment, the subject has a complex perianal fistula that is refractory to treatment with an immune modulating agent for a period of at least about 3 months. Suitable immune modulating agents will be familiar to persons skilled in the art, an illustrative example of which includes a tumour necrosis factor-α (TNF-α) inhibitor, such as an anti-TNF-α antibody. In an embodiment, the subject has a complex perianal fistula that is refractory to treatment with an anti-tumour necrosis factor-α (TNF-α) antibody for a period of at least about 3 months.

In some embodiments, the hAEC are administered to a fistula of the subject in an amount of from about 1×10⁶ to about 1×10⁹ hAECs per fistula. In some embodiments, the hAEC are administered to a fistula of the subject in an amount of from about 1×10⁶ to about 1×10⁸ hAECs per fistula. In some embodiments, the hAEC are administered to a fistula of the subject in an amount of from about 1×10⁶ to about 1×10⁷ hAECs per fistula. In another embodiment, the hAEC are administered to a fistula of the subject in an amount of from about 10×10⁶ to about 80×10⁶ hAECs per fistula. In another embodiment, the hAEC are administered to a fistula of the subject in an amount of from about 30×10⁶ to about 50×10⁶ hAECs per fistula. In yet another embodiment, the hAEC are administered to a fistula of the subject in an amount of about 40×10⁶ hAECs per fistula. In another embodiment, the hAEC are administered to a fistula of the subject in an amount of about 120×10⁶ hAECs per fistula.

In an embodiment, the subject is a human.

In an embodiment, the hAECs are autologous or allogenic or xenogeneic to the subject. In another embodiment, the hAECs are allogeneic to the subject.

In some embodiments, the hAECs are immortalised hAECs.

In some embodiments, following said screening and selection, the hAECs are cryopreserved and subsequently thawed immediately prior to administration to the subject.

In another embodiment, the subject has perianal fistulising Crohn's Disease and said treatment provides a reduction in active fistula number and/or size at 24 weeks following administration of the hAECs. In yet another embodiment, the subject has perianal fistulising Crohn's Disease and said treatment provides a reduction in active fistula number and/or size at 52 weeks following administration of the hAECs. In yet another embodiment, the subject has perianal fistulising Crohn's Disease and said treatment provides a reduction in active fistula number and/or size at 104 weeks following administration of the hAECs.

In another aspect, there is provided, human amnion epithelial cells (hAECs) for use in the treatment of a fistula in a subject, wherein said treatment comprises administering to a subject in need thereof a therapeutically effective amount of the hAECs, wherein prior to administration to the subject, the hAECs are screened and selected for their ability to suppress T cell activity.

In another aspect, there is provided, human amnion epithelial cells (hAECs) for use in the treatment of inflammatory bowel disease in a subject, wherein said treatment comprises administering to a subject in need thereof a therapeutically effective amount of the hAECs, wherein prior to administration to the subject, the hAECs are screened and selected for their ability to suppress T cell activity.

In some embodiments, the inflammatory bowel disease is ulcerative colitis. In some embodiments, the inflammatory bowel disease is Crohn's Disease. In some embodiments, the subject has perianal fistulising Crohn's Disease.

In some embodiments, the hAECs are screened and selected for their ability to suppress T cell proliferation by at least about 25%.

In some embodiments, the subject has a complex perianal fistula. In some embodiments, the subject has one or more of intersphincteric, trans-sphincteric; extra-sphincteric; and supra-sphincteric fistulas. In some embodiments, the subject has a rectovaginal fistula.

In a particular embodiment, the subject has a complex perianal fistula that is refractory to treatment with an immune modulating agent for a period of at least about 3 months. Suitable immune modulating agents will be familiar to persons skilled in the art, an illustrative example of which includes a tumour necrosis factor-α (TNF-α) inhibitor, such as an anti-TNF-α antibody. In an embodiment, the subject has a complex perianal fistula that is refractory to treatment with an anti-tumour necrosis factor-α (TNF-α) antibody for a period of at least about 3 months.

In some embodiments, the hAEC are administered to a fistula of the subject in an amount of from about 1×10⁶ to about 1×10⁹ hAECs per fistula. In some embodiments, the hAEC are administered to a fistula of the subject in an amount of from about 1×10⁶ to about 1×10⁸ hAECs per fistula. In some embodiments, the hAEC are administered to a fistula of the subject in an amount of from about 1×10⁶ to about 1×10⁷ hAECs per fistula. In another embodiment, the hAEC are administered to a fistula of the subject in an amount of from about 10×10⁶ to about 80×10⁶ hAECs per fistula. In another embodiment, the hAEC are administered to a fistula of the subject in an amount of from about 30×10⁶ to about 50×10⁶ hAECs per fistula. In yet another embodiment, the hAEC are administered to a fistula of the subject in an amount of about 40×10⁶ hAECs per fistula. In another embodiment, the hAEC are administered to a fistula of the subject in an amount of about 120×10⁶ hAECs per fistula.

In an embodiment, the subject is a human.

In an embodiment, the hAECs are autologous or allogenic or xenogeneic to the subject. In another embodiment, the hAECs are allogeneic to the subject.

In some embodiments, the hAECs are immortalised hAECs.

In some embodiments, following said screening and selection, the hAECs are cryopreserved and subsequently thawed immediately prior to administration to the subject.

In another embodiment, the subject has perianal fistulising Crohn's Disease and said treatment provides a reduction in active fistula number and/or size at 24 weeks following administration of the hAECs. In yet another embodiment, the subject has perianal fistulising Crohn's Disease and said treatment provides a reduction in active fistula number and/or size at 52 weeks following administration of the hAECs. In yet another embodiment, the subject has perianal fistulising Crohn's Disease and said treatment provides a reduction in active fistula number and/or size at 104 weeks following administration of the hAECs.

### BRIEF DESCRIPTION OF THE DRAWINGS

Embodiments of the disclosure are described herein, by way of non-limiting example only, with reference to the following drawings.

FIG. 1 is a schematic depicting the study outline.

FIG. 2 is a graph depicting the change in perianal disease activity index, comparing baseline and week 24 after hAEC injection. Asterisk in the graph denotes a patient that underwent week 24 PDAI within days after an urgent examination under anaesthesia for investigation of perianal pain. No abscess was found.

FIG. 3 is a graph depicting the change in modified van Assche Index, comparing baseline and week 24 after hAEC injection.

FIG. 4 is a graph depicting the change in perianal disease activity index, comparing baseline and week 52 after hAEC injection (n=8 of total of 10 to be assessed). Paired t-test showed a mean reduction of 5.5 points (95% CI −8.90, −2.10, p=0.007) at week 52 compared with baseline.

FIG. 5 is a graph depicting the change in modified van Assche Index, comparing baseline and week 24 after hAEC injection (n=5 of total of 10 to be assessed). Paired t-test showed a mean reduction of 1.9 points (95% CI −3.50, −0.22, p=0.035) at week 52 compared with baseline.

FIG. 6 depicts the change in PDAI, comparing baseline and week 24 (FIG. 6A) and week 52 (FIG. 6B) after hAEC injection (n=10). Paired t-tests showed a mean reduction of 6.5 points (95% CI −9.0, −4.0, p=0.0002) at week 24 compared with baseline; and a mean reduction of 6.1 points (95% CI −8.8, −3.4, p=0.0007) at week 52 compared with baseline.

FIG. 7 depicts the change in mVAI, comparing baseline and week 24 (FIG. 7A) and week 52 (FIG. 7B) after hAEC injection (n=10). Paired t-test showed a mean reduction of 2.3 points (95% CI −3.9, −0.6, p=0.012) at week 24 compared with baseline; and a mean reduction of 2.5 points (95% CI −4.5, −0.4, p=0.023) at week 52 compared with baseline.

FIG. 8 depicts the change in MAGNIFI-CD, comparing baseline and week 24 (FIG. 8A) and week 52 (FIG. 8B) after hAEC injection (n=10). Paired t-tests showed a mean reduction of 1.9 points (95% CI −3.7, −0.1, p=0.043) at week 24 compared with baseline; and a mean reduction of 2.9 points (95% CI −5.4, −0.4, p=0.027) at week 52 compared with baseline.

FIG. 9 depicts the change in sIBDQ, comparing baseline and week 24 after hAEC injection (n=10). Paired t-test showed a mean difference of 6 points (95% CI −1.7, 13.7, p=0.110) at week 24 compared with baseline.

FIG. 10 depicts the change in PDAI, comparing baseline and week 104 after hAEC injection (n=10). Paired t-test showed a mean difference of 6.3 points (95% CI −8.8, −3.8, p=0.0003) at week 104 compared with baseline.

FIG. 11 depicts the change in Fistula Assessment Score (FAS), comparing baseline and week 24 (FIG. 11A), week 52 (FIG. 11B) and week 104 (FIG. 11C) after hAEC injection (n=10). Paired t-tests showed a mean difference of 2.9 points (95% CI −4.50 to −1.30, p=0.003) at week 24 compared with baseline; a mean difference of 2.5 points (95% CI −4.36 to −0.65, p=0.014) at week 52 compared with baseline; and a mean difference of 2.8 points (95% CI −4.58 to −1.02, p=0.006) at week 104 compared with baseline.

FIG. 12 depicts a Fistula Assessment form, used to assess and score fistula symptoms.

FIG. 13 depicts T cell proliferation potency profile of hAECs isolated from 31 placentae. Flow cytometry was used to assess the suppression of T cell proliferation. The immunomodulatory potential can vary from different donor hAECs, from high suppressive proliferation index (e.g. proliferative index of 1) to less suppressive (e.g. proliferative index of 2).

### DETAILED DESCRIPTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

As used herein, “and/or” refers to and encompasses any and all possible combinations of one or more of the associated listed items, as well as the lack of combinations when interpreted in the alternative (or).

The terms “active agent” and “therapeutic agent” are used interchangeably herein and refer to agents that prevent, reduce or ameliorate at least one symptom of a disease or disorder.

As used herein, the terms “administration”, “administering” and the like, refer to routes of administration include local administration and systemic administration. The routes of administration refer to, but are not limited to, respiratorally, intratracheally, nasopharyngeally, intravenously, intraperitoneally, intrathoracically, subcutaneously, intracranially, intradermally, intramuscularly, intraoccularly, intrathecally, intracereberally, intranasally, rectally, topically, patch, bandage and implant. In an embodiment the hAEC are locally injected or administered. In the context of treating a fistula, it is to be understood that by “locally” injected/administered is meant that the hAEC are administered to the fistula. Thus, in an embodiment, the hAEC are locally administered to the fistula.

The terms “administration concurrently” or “administering concurrently” or “co-administering” and the like refer to the administration of a single composition containing two or more agents, or the administration of each agent as separate compositions and/or delivered by separate routes either contemporaneously or simultaneously or sequentially within a short enough period of time that the effective result is equivalent to that obtained when all such agents are administered as a single composition. By “simultaneously” is meant that the agents are administered at substantially the same time, and desirably together in the same formulation. By “contemporaneously” it is meant that the agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The agents may be administered in either order. The term “sequentially” as used herein means that the agents are administered in sequence, for example at an interval or intervals of minutes, hours, days or weeks. If appropriate the agents may be administered in a regular repeating cycle.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The term a “disease” or “condition” also includes a “disorder”. All such aspects are enabled within the width of the present invention.

By “effective amount”, in the context of treating a disease or condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the age, health and physical condition of the individual to be treated and whether symptoms of disease are apparent, the taxonomic group of individual to be treated, the formulation of the composition, the assessment of the medical situation, and other relevant factors. Optimal dosing schedules can be calculated from measurements of drug accumulation in the body of the subject. Optimum dosages may vary depending on the relative potency in an individual subject, and can generally be estimated based on EC50 values found to be effective in in vitro and in vivo animal models. Persons of ordinary skill can easily determine optimum dosages, dosing methodologies and repetition rates. It is expected that the amount will fall in a relatively broad range that can be determined through routine trials.

By “isolated” is meant material that is substantially or essentially free from components that normally accompany it in its native state.

The terms “subject”, “patient” and “individual” used interchangeably herein, refer to any subject, particularly a vertebrate subject, and even more particularly a mammalian subject, (e.g. human).

As used herein, the terms “treatment”, “treating”, and the like, refer to obtaining a desired pharmacologic and/or physiologic effect in a subject in need of treatment, including a subject who has a fistula or inflammatory bowel disease (IBD). In an embodiment, the subject has one or more fistulas. In an embodiment, the subject has Crohn's Disease. In an embodiment, the subject has perianal fistulising Crohn's Disease. In some embodiments, the subject has a complex perianal fistula. The term “treatment”, as used herein, includes ameliorating or preventing one or more symptoms or effects (e.g. consequences) arising from a fistula. This may suitably include closure of one or more, or all external fistula orifices without abscess on MRI. This may also suitably include a reduction in fistula number or size. The treatment may result in a complete response, which may be defined by the absence of fistula drainage with closure of treated external openings and absence of collection (>2 cm diameter) on MRI. Alternatively, the treatment may result in a partial response, which may be defined by closure of >50% of all treated external openings or >50% reduction of fistula diameter on MRI (if a single fistula), also in the absence of an abscess on MRI. A complete response or partial response can also be assessed using any one of the disease scoring indices or active disease markers known to persons skilled in the art, illustrative examples of which are disclosed elsewhere herein.

In an embodiment, the method described herein provides a mean reduction in PDAI score of from about 2 to about 15 points (e.g., 2, 2.5, 3, 3.5, 4, 4.5, 5, 5.5, 6, 6.5, 7, 7.5 8, 8.5, 9. 9.5, 10, 11, 12, 13, 14 or 15 points), preferably from about 2 to about 14 points, preferably from about 2 to about 12 points, preferably from about 2 to about 10 points, 3 to about 8 points, preferably from about 4 to about 8 points, preferably from about 5 to about 7 points, preferably from about 6 to about 7 points, or more preferably about 6.5 points at 24 weeks following administration of the hAEC to the subject, compared to baseline.

In an embodiment, after treatment, the subject has a PDAI score of less than about 8, preferably less than about 7, preferably less than about 6, preferably less than about 5, or more preferably less than about 4, compared to baseline. In an embodiment, the subject is seton free at week 24.

In an embodiment, the method described herein provides a mean reduction in mVAI score of from about 1 to about 20 points (e.g., 1, 1.5, 2, 2.5, 3, 3.5, 4, 4.5, 5, 5.5, 6, 6.5, 7, 7.5 8, 8.5, 9. 9.5, 10, 11, 12, 13, 14, 15, 16, 17, 18, 19 or 20 points), preferably from about 1 to about 15, preferably from about 1 to about 12, preferably from about 1 to about 10, preferably from about 1 to about 8, preferably from about 1 to about 5 points, preferably from about 1 to about 4 points, preferably from about 1 to about 3 points, preferably from about 2 to about 3 points, or more preferably about 2.3 points at 24 weeks following administration of the hAEC to the subject, compared to baseline.

In an embodiment, the method described herein provides a mean reduction in MAGNIFI-CD score of from about 1 to about 0 points (e.g., 1, 1.5, 2, 2.5, 3, 3.5, 4, 4.5, 5, 5.5, 6, 6.5, 7, 7.5 8, 8.5, 9. 9.5, 10, 11, 12, 13, 14, 15, 16, 17, 18, 19, 20, 22, 24, 26, 28, 30, 32, 34, 36, 38 or 40 points) preferably from about 1 to about 35, preferably from about 1 to about 30, preferably from about 1 to about 25, preferably from about 1 to about 20, preferably from about 1 to about 15, preferably from about 1 to about 12, preferably from about 1 to about 10, preferably from about 1 to about 8, preferably from about 1 to about 5 points, preferably from about 1 to about 4 points, preferably from about 1 to about 3 points, preferably from about 1 to about 2 points, or more preferably about 2 points (e.g., 1.9 points) at 24 weeks following administration of the hAEC to the subject, compared to baseline.

In an embodiment, the method described herein provides a mean difference in sIBDQ score of from about 2 to about 50 points (e.g., 2, 2.5, 3, 3.5, 4, 4.5, 5, 5.5, 6, 6.5, 7, 7.5 8, 8.5, 9. 9.5, 10, 10.5, 11, 11.5, 12, 12.5, 13, 13.5, 14, 14.5, 15, 15.5, 16, 16.5, 17, 17.5, 18, 18.5, 19, 19.5, 20, 25, 30, 35, 40, 45 or 50 points), preferably from about 2 to about 45 points, preferably from about 2 to about 40 points, preferably from about 2 to about 35 points, preferably from about 2 to about 30 points, preferably from about 2 to about 25 points, preferably from about 2 to about 20 points, preferably from about 2 to about 15 points, preferably from about 2 to about 14 points, preferably from about 2 to about 13 points, preferably from about 2 to about 12 points, preferably from about 2 to about 11 points, preferably from about 2 to about 10 points, preferably from about 3 to about 8 points, preferably from about 4 to about 8 points, preferably from about 5 to about 7 points, or more preferably about 6.0 points at 24 weeks following administration of the hAEC to the subject, compared to baseline; or preferably from about 10 to about 18 points, preferably from about 11 to about 17 points, preferably from about 13 to about 17 points, preferably from about 14 to about 17 points, preferably from about 15 to about 17 points, preferably from about 15 to about 16 points, or more preferably about 16 points (e.g., about 15.8 points) at 24 weeks following administration of the hAEC to the subject, compared to baseline.

In an embodiment, the method described herein provides a mean reduction in FAS score of from about 1 to about 12 points (e.g., 1, 2, 3, 4, 5, 6, 7, 8, 9, 10, 11 or 12 points), preferably from about 1 to about 11 points, preferably from about 1 to about 10 points, preferably from about 1 to about 9 points, preferably from about 1 to about 8 points, preferably from about 1 to about 7 points, preferably from about 1 to about 6 points, preferably from about 1 to about 5 points, preferably from about 1 to about 4 points, preferably from about 1 to about 3 points, preferably from about 1 to about 2 points, preferably from about 2 to about 12 points, preferably from about 2 to about 11 points, preferably from about 2 to about 10 points, preferably from about 2 to about 9 points, preferably from about 2 to about 8 points, preferably from about 2 to about 7 points, preferably from about 2 to about 6 points, preferably from about 2 to about 5 points, preferably from about 2 to about 4 points, or preferably from about 2 to about 3 points, at 24 weeks following administration of the hAEC to the subject, compared to baseline; or more preferably about 3 points (e.g., about 2.8 points) at 24 weeks following administration of the hAEC to the subject, compared to baseline.

In an embodiment, the method described herein provides a mean reduction in PDAI score of from about 2 to about 15 points (e.g., 2, 2.5, 3, 3.5, 4, 4.5, 5, 5.5, 6, 6.5, 7, 7.5 8, 8.5, 9. 9.5, 10, 11, 12, 13, 14 or 15 points), preferably from about 2 to about 14 points, preferably from about 2 to about 12 points, preferably from about 2 to about 10 points, 3 to about 8 points, preferably from about 4 to about 8 points, preferably from about 5 to about 7 points, preferably from about 6 to about 7 points, or more preferably about 6.5 points at 52 weeks following administration of the hAEC to the subject, compared to baseline.

In an embodiment, after treatment, the subject has a PDAI score of less than about 8, preferably less than about 7, preferably less than about 6, preferably less than about 5, or more preferably less than about 4, compared to baseline. In an embodiment, the subject is seton free at week 52.

In an embodiment, the method described herein provides a mean reduction in mVAI score of from about 1 to about 20 points (e.g., 1, 1.5, 2, 2.5, 3, 3.5, 4, 4.5, 5, 5.5, 6, 6.5, 7, 7.5 8, 8.5, 9. 9.5, 10, 11, 12, 13, 14, 15, 16, 17, 18, 19 or 20 points), preferably from about 1 to about 15, preferably from about 1 to about 12, preferably from about 1 to about 10, preferably from about 1 to about 8, preferably from about 1 to about 5 points, preferably from about 1 to about 4 points, preferably from about 1 to about 3 points, preferably from about 2 to about 3 points, or more preferably about 2.3 points at 52 weeks following administration of the hAEC to the subject, compared to baseline.

In an embodiment, the method described herein provides a mean reduction in MAGNIFI-CD score of from about 1 to about 0 points (e.g., 1, 1.5, 2, 2.5, 3, 3.5, 4, 4.5, 5, 5.5, 6, 6.5, 7, 7.5 8, 8.5, 9. 9.5, 10, 11, 12, 13, 14, 15, 16, 17, 18, 19, 20, 22, 24, 26, 28, 30, 32, 34, 36, 38 or 40 points) preferably from about 1 to about 35, preferably from about 1 to about 30, preferably from about 1 to about 25, preferably from about 1 to about 20, preferably from about 1 to about 15, preferably from about 1 to about 12, preferably from about 1 to about 10, preferably from about 1 to about 8, preferably from about 1 to about 5 points, preferably from about 1 to about 4 points, preferably from about 1 to about 3 points, preferably from about 1 to about 2 points, or more preferably about 2 points (e.g., 1.9 points) at 52 weeks following administration of the hAEC to the subject, compared to baseline.

In an embodiment, the method described herein provides a mean difference in SIBDQ score of from about 2 to about 50 points (e.g., 2, 2.5, 3, 3.5, 4, 4.5, 5, 5.5, 6, 6.5, 7, 7.5 8, 8.5, 9. 9.5, 10, 10.5, 11, 11.5, 12, 12.5, 13, 13.5, 14, 14.5, 15, 15.5, 16, 16.5, 17, 17.5, 18, 18.5, 19, 19.5, 20, 25, 30, 35, 40, 45 or 50 points), preferably from about 2 to about 45 points, preferably from about 2 to about 40 points, preferably from about 2 to about 35 points, preferably from about 2 to about 30 points, preferably from about 2 to about 25 points, preferably from about 2 to about 20 points, preferably from about 2 to about 15 points, preferably from about 2 to about 14 points, preferably from about 2 to about 13 points, preferably from about 2 to about 12 points, preferably from about 2 to about 11 points, preferably from about 2 to about 10 points, preferably from about 3 to about 8 points, preferably from about 4 to about 8 points, preferably from about 5 to about 7 points, or more preferably about 6.0 points at 24 weeks following administration of the hAEC to the subject, compared to baseline; or preferably from about 10 to about 18 points, preferably from about 11 to about 17 points, preferably from about 13 to about 17 points, preferably from about 14 to about 17 points, preferably from about 15 to about 17 points, preferably from about 15 to about 16 points, or more preferably about 16 points (e.g., about 15.8 points) at 52 weeks following administration of the hAEC to the subject, compared to baseline.

In an embodiment, the method described herein provides a mean reduction in FAS score of from about 1 to about 12 points (e.g., 1, 2, 3, 4, 5, 6, 7, 8, 9, 10, 11 or 12 points), preferably from about 1 to about 11 points, preferably from about 1 to about 10 points, preferably from about 1 to about 9 points, preferably from about 1 to about 8 points, preferably from about 1 to about 7 points, preferably from about 1 to about 6 points, preferably from about 1 to about 5 points, preferably from about 1 to about 4 points, preferably from about 1 to about 3 points, preferably from about 1 to about 2 points, preferably from about 2 to about 12 points, preferably from about 2 to about 11 points, preferably from about 2 to about 10 points, preferably from about 2 to about 9 points, preferably from about 2 to about 8 points, preferably from about 2 to about 7 points, preferably from about 2 to about 6 points, preferably from about 2 to about 5 points, preferably from about 2 to about 4 points, or preferably from about 2 to about 3 points, at 24 weeks following administration of the hAEC to the subject, compared to baseline; or more preferably about 3 points (e.g., about 2.8 points) at 52 weeks following administration of the hAEC to the subject, compared to baseline.

In an embodiment, the method described herein provides a mean reduction in PDAI score of from about 2 to about 15 points (e.g., 2, 2.5, 3, 3.5, 4, 4.5, 5, 5.5, 6, 6.5, 7, 7.5 8, 8.5, 9. 9.5, 10, 11, 12, 13, 14 or 15 points), preferably from about 2 to about 14 points, preferably from about 2 to about 12 points, preferably from about 2 to about 10 points, 3 to about 8 points, preferably from about 4 to about 8 points, preferably from about 5 to about 7 points, preferably from about 6 to about 7 points, or more preferably about 6.5 points at 104 weeks following administration of the hAEC to the subject, compared to baseline.

In an embodiment, after treatment, the subject has a PDAI score of less than about 8, preferably less than about 7, preferably less than about 6, preferably less than about 5, or more preferably less than about 4, compared to baseline. In an embodiment, the subject is seton free at week 104.

In an embodiment, the method described herein provides a mean reduction in mVAI score of from about 1 to about 20 points (e.g., 1, 1.5, 2, 2.5, 3, 3.5, 4, 4.5, 5, 5.5, 6, 6.5, 7, 7.5 8, 8.5, 9. 9.5, 10, 11, 12, 13, 14, 15, 16, 17, 18, 19 or 20 points), preferably from about 1 to about 15, preferably from about 1 to about 12, preferably from about 1 to about 10, preferably from about 1 to about 8, preferably from about 1 to about 5 points, preferably from about 1 to about 4 points, preferably from about 1 to about 3 points, preferably from about 2 to about 3 points, or more preferably about 2.3 points at 104 weeks following administration of the hAEC to the subject, compared to baseline.

In an embodiment, the method described herein provides a mean reduction in MAGNIFI-CD score of from about 1 to about 0 points (e.g., 1, 1.5, 2, 2.5, 3, 3.5, 4, 4.5, 5, 5.5, 6, 6.5, 7, 7.5 8, 8.5, 9. 9.5, 10, 11, 12, 13, 14, 15, 16, 17, 18, 19, 20, 22, 24, 26, 28, 30, 32, 34, 36, 38 or 40 points) preferably from about 1 to about 35, preferably from about 1 to about 30, preferably from about 1 to about 25, preferably from about 1 to about 20, preferably from about 1 to about 15, preferably from about 1 to about 12, preferably from about 1 to about 10, preferably from about 1 to about 8, preferably from about 1 to about 5 points, preferably from about 1 to about 4 points, preferably from about 1 to about 3 points, preferably from about 1 to about 2 points, or more preferably about 2 points (e.g., 1.9 points) at 104 weeks following administration of the hAEC to the subject, compared to baseline.

In an embodiment, the method described herein provides a mean difference in sIBDQ score of from about 2 to about 50 points (e.g., 2, 2.5, 3, 3.5, 4, 4.5, 5, 5.5, 6, 6.5, 7, 7.5 8, 8.5, 9. 9.5, 10, 10.5, 11, 11.5, 12, 12.5, 13, 13.5, 14, 14.5, 15, 15.5, 16, 16.5, 17, 17.5, 18, 18.5, 19, 19.5, 20, 25, 30, 35, 40, 45 or 50 points), preferably from about 2 to about 45 points, preferably from about 2 to about 40 points, preferably from about 2 to about 35 points, preferably from about 2 to about 30 points, preferably from about 2 to about 25 points, preferably from about 2 to about 20 points, preferably from about 2 to about 15 points, preferably from about 2 to about 14 points, preferably from about 2 to about 13 points, preferably from about 2 to about 12 points, preferably from about 2 to about 11 points, preferably from about 2 to about 10 points, preferably from about 3 to about 8 points, preferably from about 4 to about 8 points, preferably from about 5 to about 7 points, or more preferably about 6.0 points at 24 weeks following administration of the hAEC to the subject, compared to baseline; or preferably from about 10 to about 18 points, preferably from about 11 to about 17 points, preferably from about 13 to about 17 points, preferably from about 14 to about 17 points, preferably from about 15 to about 17 points, preferably from about 15 to about 16 points, or more preferably about 16 points (e.g., about 15.8 points) at 104 weeks following administration of the hAEC to the subject, compared to baseline.

In an embodiment, the method described herein provides a mean reduction in FAS score of from about 1 to about 12 points (e.g., 1, 2, 3, 4, 5, 6, 7, 8, 9, 10, 11 or 12 points), preferably from about 1 to about 11 points, preferably from about 1 to about 10 points, preferably from about 1 to about 9 points, preferably from about 1 to about 8 points, preferably from about 1 to about 7 points, preferably from about 1 to about 6 points, preferably from about 1 to about 5 points, preferably from about 1 to about 4 points, preferably from about 1 to about 3 points, preferably from about 1 to about 2 points, preferably from about 2 to about 12 points, preferably from about 2 to about 11 points, preferably from about 2 to about 10 points, preferably from about 2 to about 9 points, preferably from about 2 to about 8 points, preferably from about 2 to about 7 points, preferably from about 2 to about 6 points, preferably from about 2 to about 5 points, preferably from about 2 to about 4 points, or preferably from about 2 to about 3 points, at 24 weeks following administration of the hAEC to the subject, compared to baseline; or more preferably about 3 points (e.g., about 2.8 points) at 104 weeks following administration of the hAEC to the subject, compared to baseline.

By “treatment” is meant ameliorating or preventing one or more symptoms or effects (e.g. consequences) of fistulas or the IBD. In particular examples, treatment includes ameliorating or preventing clinical disease indices of fistulas or the IBD (e.g., the number of perianal fistulas). Reference to “treatment”, “treat” or “treating” does not necessarily mean to reverse or prevent any or all symptoms or effects of the fistula or the IBD. For example, the subject may ultimately suffer one or more symptoms or effects, but the number and/or severity of the symptoms or effects is reduced and/or the quality of life is improved compared to prior to treatment.

Each embodiment described herein is to be applied mutatis mutandis to each and every embodiment unless specifically stated otherwise.

### 2. Fistulas

Fistulas are abnormal connection between two body parts, such as an organ or blood vessel and another structure that normally are not connected. Fistulas often result in abnormal flow of fluid or material from one space to the other.

A perianal fistula is a chronic abnormal communication between the anal canal and the perianal skin. The predominant perianal fistulas are cryptoglandular anal fistulas arising from an inflammation of the anal glands, and perianal fistulas are associated with and/or the result of Crohn's Disease. While cryptoglandular and Crohn's perianal fistulas may have distinct etiologies, they share a similar pathophysiology.

An anovaginal or rectovaginal fistula is a connection that should not normally exist between the lower part of the large intestine (the rectum or anus) and the vagina. A colovaginal fistula joins the space in the colon to that in the vagina.

The current treatment for a fistula varies depending on the cause and extent of the fistula, but often involves surgical intervention combined with antibiotic therapy. In some cases, the fistula is temporarily covered using a fibrin glue or plug. A catheter may be required to drain a fistula.

In one aspect, there is provided herein methods for treating a fistula in a subject, the method comprising administering to a subject in need thereof a therapeutically effective amount of human amnion epithelial cells (hAECs). In an embodiment, the hAEC are locally administered to the fistula.

### 3. Inflammatory Bowel Disease

Inflammatory bowel disease is a complex idiopathic condition of the gastrointestinal tract that is increasingly prevalent in industrialized countries, with an estimated more than one million sufferers in the United States alone. There are two main subtypes of inflammatory bowel disease, Crohn's disease and ulcerative colitis. Crohn's disease can occur at any point along the lower gastrointestinal tract, although it primarily affects the ileum and the large intestine. Ulcerative colitis is mostly limited to the colon and can predispose individuals to colitis-associated cancer, typically colorectal cancer.

The etiologies of Crohn's disease and ulcerative colitis remain to be fully elucidated, however they are considered to be inflammatory conditions of the intestinal mucosa, characterized by the alteration and dysregulation of immune response towards commensal microbiota of the gastrointestinal tract. Symptomatically, the diseases are associated with abdominal pain, diarrhea (often bloody), and varied other clinical manifestations such as arthritis and uveitis.

The at least one symptom associated with the inflammatory bowel disease may be a clinical or physiological symptom including, for example, diarrhoea, poor stool consistency, faecal blood presence, abdominal pain, ulceration of the epithelial lining of the small intestine, large intestine or colon, or elevated expression of one or more pro-inflammatory cytokines relative to the level of expression observed in individuals not suffering from the inflammatory bowel disease. Those skilled in the art will readily appreciate that the scope of the present disclosure should not be limited by reference these exemplary symptoms, and there are other symptoms of inflammatory bowel disease that will be encompassed by the present disclosure.

The inflammatory bowel diseases to which the present disclosure relate include, but is not limited to, ulcerative colitis, Crohn's disease, ischemic colitis, enterocolitis, antibiotic-associated hemorrhagic colitis (AAHC), microscopic colitis and pouchitis. In an embodiment, the inflammatory bowel disease is Crohn's disease. In an embodiment, the inflammatory bowel disease is perianal fistulising Crohn's Disease.

In an embodiment, the subject has a rectovaginal fistula. Rectovaginal fistulas are abnormal epithelial-lined connections between the rectum and vagina. Rectovaginal fistulas can occur as a childbirth related injury, but the second most common cause of rectovaginal fistula is Crohn's Disease, as Crohn's disease increases the risk of rectovaginal fistula formation.

In an embodiment, the subject has a perianal fistula, including a complex perianal fistula.

A perianal fistula typically refers to an abnormal connection that develops between the anal canal and the perianal skin, usually from the result of infection, causing a collection of pus in tissue and resultant abscesses and lesions. Perianal fistulas can include intersphincteric, trans-sphincteric; extra-sphincteric; or supra-sphincteric fistulas.

Complex perianal Crohn's fistula, as defined by the American Gastroenterological Association (AGA), requires at least one of the following: high intersphincteric or trans-sphincteric fistulas, supra-sphincteric fistulas, two or more external fistula openings or any associated collections; inactive or mildly active luminal rectal inflammation (on endoscopic assessment based on the absence of ulcers), and active fistulas with a Perianal Disease Activity Index (PDAI) score greater than four. The methods of the present disclosure may inhibit inflammation associated with the inflammatory bowel disease. The term “inhibit” and variations thereof such as “inhibition”, “inhibits”, “reduces”, “reducing” and the like, are used interchangeably herein to denote an improvement (i.e., reduction) in severity, including of inflammation associated with the inflammatory bowel disease.

The person skilled in the art would be familiar with the tools and methods of measuring progression of disease or effectiveness of treatment. Such methods include determining original and modified Van Assche indices, which are a semiquantitative scoring systems used to assess the severity and disease response, in particular in perianal fistulizing Crohn's Disease. Modified Van Assche index is a magnetic resonance imaging-based scoring system, which takes into account T1 hyperintensity and infiltrate to the score. A high MRI-based score was confirmed to be associated with more severe clinical behavior. Another method of measuring severity of disease includes the Perianal disease activity index (PDAI).

### 4. Human Amnion Epithelial Cells

The term “human amnion epithelial cells” (hAEC) typically refers to cells extracted or derived from the innermost lining of the placenta. Methods of hAEC isolation for clinical use would be known to the person skilled in the art, for example, the protocols and procedures disclosed in Murphy et al. 2010 Curr Protoc Stem Cell Biol Chapter 1: Unit 1E 6. hAEC may be derived from pre-term or term placentas. In some embodiments, the hAEC may be maintained as an immortalized cell line, and further hAEC be derived from immortalized hAEC.

hAECs may be identified by their expression of cell surface markers, illustrative examples of which include Epithelial Cell Adhesion Marker (EpCAM), CK-7, stage-specific embryonic antigen-3 and 4 (SSEA-3 and SSEA-4), octamer-binding transcription factor-4 (OCT-4), and tumor rejection antigen1-60 and 1-80 (TRA1-60 and TRA1-80) (Miki et al. (2005) Stem Cells 23 (10): 1549-59). hAECs may also be characterized by negative for mesenchymal stem cell markers (CD90, CD105) or absence of CD45. Although hAECs possess stem cell-like characteristics, they typically do not exhibit unlimited proliferation due to the lack of telomerase activity. hAECs also have multi-potency differentiation potential and low expression levels of human leukocyte antigens.

In some embodiments, the hAECs may be derived from one or more pre-term placentas. In some embodiments, the hAECs may be derived from one or more term placentas.

In some embodiments, the screening and selecting hAECs for cell viability and/or their ability to suppress T cell activity may be undertaken hours, days or months prior to administration to the subject in need thereof. For example, the hAECs may be suitably screened and selected from a depository of hAECs, where the hAECs have been catalogued on the basis of, for example, information collected on the ability of the hAEC to suppress T cell activity, including in accordance with the methods described herein. Thus, included herein are embodiments in which the methods described herein do not require an active step of screening and selecting hAECs that fulfill the desired functional criteria, such as cell viability and T cell activity, immediately prior to administration to a subject in need thereof. In some embodiments, the hAECs that have been screened and selected to fulfill the desired cell viability and/or T cell activity criteria. In some embodiments, the selected hAECs are cryopreserved and thawed for administration to the subject. In some embodiments, the screening and selecting of hAECs for cell viability and/or their ability to suppress T cell activity is conducted prior to cryopreservation.

In some embodiments, the hAECs that are screened and selected for cell markers, cell viability and/or their ability to suppress T cell proliferation are a subset of a population of hAECs to be administered to the subject. In an embodiment, the hAEC cells are screened and selected based on their EpCAM, CD90, CD105, CD45 and/or CK7 expression profiles. In an embodiment, the hAECs are EpCAM positive and negative for MSC markers, CD90 and CD105. In an embodiment, the hAECs are negative for non-epithelial cells markers CD105 and CD45. In an embodiment, the hAECs are negative for CD45 and CK7 positive. In some embodiments, the hAEC were screened and selected for their ability to suppress T cell activity.

In some embodiments, the hAECs were screened and selected for cell viability and their ability to suppress T cell activity. In some embodiments, the hAECs were screened and selected for their ability to suppress T cell proliferation. In some embodiments, the hAECs were screened and selected for their ability to suppress T cell proliferation by at least about 25%. In some embodiments, the hAECs are screened and selected for cell viability and their ability to suppress T cell activity. In some embodiments, the hAECs are screened and selected for their ability to suppress T cell proliferation. In some embodiments, the hAECs are screened and selected for their ability to suppress T cell proliferation by at least about 25%.

The amount of hAECs to be administered to a subject will vary depending on, for example, on disease severity of the disease, the treatment goal, the route of administration, and the individual to be treated, and can be determined by methods well known to those skilled in the art. The exact dosage may suitably be determined on an individual basis, however, in some cases, the hAEC may be administered to a fistula of the subject in an amount of from about 1×10⁶ to about 1×10⁹ hAECs per fistula. In some embodiments, the hAEC are administered to a fistula of the subject in an amount of from about 1×10⁶ to about 1×10⁹ hAECs per fistula. In some embodiments, the hAEC are administered to a fistula of the subject in an amount of from about 1×10⁶ to about 1×10⁸ hAECs per fistula. In some embodiments, the hAEC are administered to a fistula of the subject in an amount of from about 1×10⁶ to about 1×10⁷ hAECs per fistula. In another embodiment, the hAEC are administered to a fistula of the subject in an amount of from about 10×10⁶ to about 80×10⁶ hAECs per fistula. In another embodiment, the hAEC are administered to a fistula of the subject in an amount of from about 30×10⁶ to about 50×10⁶ hAECs per fistula. In yet another embodiment, the hAEC are administered to a fistula of the subject in an amount of about 40×10⁶ hAECs per fistula. In another embodiment, the hAEC are administered to a fistula of the subject in an amount of about 120×10⁶ hAECs per fistulas.

The hAECs can be administrated as a single dose or multiple doses. Where the hAECs are administered as multiple doses, the doses may be administered hours, days, weeks or months apart. Suitable administration intervals may be determined by persons skilled in the art and will typically depend on, for example, disease severity, treatment goal, route of administration, and the individual to be treated. For example, for more severe conditions, it might be desirable to administer multiple doses of the hAECs 1 day apart, whereas for less severe conditions, it might be desirable to administer multiple doses of the hAECs 2 or more (e.g., 2, 3, 4, 5, 6, 7, 8, etc) days apart. The doses administered can be in the same amount of hAECs each time. The doses administered can be in a varying amount of hAECs each time. The multiple doses can be administered as a form of maintenance therapy. In an embodiment, the hAECs can be administrated as a single dose. In another embodiment, the hAECs can be administrated as multiple doses. In another embodiment, the hAECs can be administrated as multiple doses at varying intervals of time.

The hAECs of the present invention can be administered in a pharmaceutical composition or pharmaceutical formulation. Embodiments of pharmaceutical compositions of the present disclosure comprise an effective amount of hAECs dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that comprises cells are known to those of skill in the art in light of the present disclosure, as exemplified by Remington: The Science and Practice of Pharmacy, 21st Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, hydrogels, biologic scaffolds, binders, excipients, disintegration agents, lubricants, sweetening agents, flavoring agents, dyes, such like materials and combinations thereof, as would be known to one of ordinary skill in the art. Except insofar as any conventional carrier is incompatible for use with hAECs and/or cells, its use in the pharmaceutical compositions is contemplated.

The hAECs will typically be formulated for administration in a pharmaceutically acceptable excipient or composition. Suitable pharmaceutically acceptable excipients and compositions will be familiar to persons skilled in the art, illustrative examples of which include phosphate buffered saline (PBS), isotonic saline, dextrose, dextran, heparin, Hartmann's solution, cell culture medium, amino acids, vitamins, sugars and/or proteins. In an embodiment, the hAECs are formulated in a pharmaceutical composition comprising albumin. In an embodiment, the hAECs are formulated in a pharmaceutical composition comprising plant albumin. In an embodiment, the hAECs are formulated in a pharmaceutical composition comprising recombinant albumin. In an embodiment, the hAECs are formulated in a pharmaceutical composition comprising ovalbumin albumin. In an embodiment, the hAECs are formulated in a pharmaceutical composition comprising lactalbumin. In an embodiment, the hAECs are formulated in a pharmaceutical composition comprising mammalian serum albumin. In an embodiment, the hAECs are formulated in a pharmaceutical composition comprising human serum albumin. In an embodiment, the hAECs are formulated in a pharmaceutical composition comprising about 1-5% human serum albumin. In another embodiment, the hAECs are formulated in a pharmaceutical composition comprising about 2.5% human serum albumin. In another embodiment, the hAECs are formulated in a pharmaceutical composition comprising about 2% human serum albumin.

The pharmaceutical formulations disclosed herein, which may conveniently be presented in unit dosage form, may be prepared according to conventional techniques well known to persons skilled in the art. Such techniques include the step of bringing into association the hAECs with the pharmaceutical carrier(s) or excipient(s). The hAEC-comprising pharmaceutical compositions of the present invention may be administered in a number of ways depending upon whether local or systemic treatment is desired and upon the area to be treated. Administration may be topical (including vaginal and rectal delivery), epidermal and transdermal, or parenteral. Parenteral administration includes intravenous, intraarterial, subcutaneous, intraperitoneal or intramuscular injections or infusion. In one embodiment, the pharmaceutical composition comprising the hAECs is locally injected at or around the fistula site. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

### 5. T Cell Suppression Activity

Methods of determining (measuring) whether an hAEC is able to suppress T cell activity will be familiar to persons skilled in the art, including those as described in Zappasodi et al 2020 Methods Enzymol. 631:42-59. Such methods can be in vitro or in vivo assays, and use one or more markers T cell activity (e.g., expression of proteins/transcripts such as cytokines and growth factors, the capacity of hAEC to activate inhibitory signalling pathways in a T cell, the capacity of hAEC to inhibit activating signalling pathways in a T cell). In an embodiment, the ability of the hAEC to suppress T cell activity is determined by exposing the hAEC to a T cell and measuring the ability of the hAEC to reduce the level of expression of T cell activation markers, illustrative examples of which will be familiar to persons skilled in the art and include lymphokines, interleukins, interferons, monokines, colony stimulating factors, and chemokines. Illustrative examples of suitable markers include IL-1, IL-2, IL-3, IL-4, IL-5, IL-6, IL-7, IL-8, IL-9, IL-10, IL-11, IL-12, IL-13, IL-15, IL-17, IFN-α, IFN-β, IFN-ε, IFN-κ, IFN-γ, IFN-ω, Granulocyte-macrophage colony-stimulating factor, macrophage colony-stimulating factor, CC chemokines, CXC chemokines, and C chemokines. In another embodiment, the hAEC are screened and selected for their ability to inhibit T cell proliferation.

Methods of determining (measuring) the suppression of T cell proliferation will be familiar to persons skilled in the art. Such methods may be suitably performed in vitro or in vivo, illustrative examples of which are described elsewhere herein. Further illustrative examples include an assessment of T cell proliferation by staining T cells with radioactive thymidine; labelled thymidine analogs (e.g. BrdU, EdU of Click-iT EdU); DNA binding stains; or a fluorescent tracking dyes, such as carboxyfluorescein succinimidyl ester (CFSE) or CellTrace™ fluorescent dyes, and monitoring dilution of the dye in daughter cells as cells divide over time. The amount of dye in each cell is halved and this produces discrete shifts or peaks in fluorescence intensity associated with each cell generation, which can be monitored using flow cytometry. Briefly, T cells derived from the peripheral blood from healthy donors are stained with a viability dye and activated with anti-CD3 and anti-CD28 antibodies, which can be suitably coated on an substrate, such as Dynabeads. Other suitable methods of activating T cells will be known to the person skilled in the art. The activated T cells are then mixed with hAECs (at a 1:10 ratio, i.e. 10,000 T cells to 100,000 hAECs; to 1:160 ratio, i.e. 10,000 T cells to 1600,000 hAECs), and kept in co-culture with the hAECs for 4 days prior to assessment of T cell proliferation, for example, by flow cytometry.

In an embodiment, the hAECs are screened and selected for their ability to inhibit or suppress T cell proliferation by at least about 25%. In another embodiment, the hAECs are screened and selected for their ability to inhibit or suppress T cell proliferation by at least about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or by at least about 100%. In an embodiment, the hAECs are screened and selected for their ability to inhibit or suppress T cell proliferation by at least 30%. In another embodiment, the hAECs are screened and selected for their ability to inhibit or suppress T cell proliferation by at least 35%. In an embodiment, the hAECs are screened and selected for their ability to inhibit or suppress T cell proliferation by at least 40%. In an embodiment, the hAECs are screened and selected for their ability to inhibit or suppress T cell proliferation by at least 45%. In an embodiment, the hAECs are screened and selected for their ability to inhibit or suppress T cell proliferation by at least 50%. In an embodiment, the hAECs are screened and selected for their ability to inhibit or suppress T cell proliferation by at least 55%. In an embodiment, the hAECs are screened and selected for their ability to inhibit or suppress T cell proliferation by at least 65%.

In an embodiment, the hAECs are screened and selected for their ability to inhibit or suppress T cell proliferation according to their T cell division index. In an embodiment, the T cell division index is the average number of cell divisions that a cell in the original population has undergone (calculated by (total number of divisions/number of cells at the start of the culture)). In another embodiment, the hAECs are screened and selected for their ability to inhibit or suppress T cell proliferation according to their expansion index. In an embodiment, the expansion index is calculated by (total number of cells/number of cells at the start of the culture). In another embodiment, the hAECs are screened and selected for their ability to inhibit or suppress T cell proliferation according to their replication index. In an embodiment, the expansion index is calculated by (total number of divided cells/cells that went into division).

In an embodiment, the hAECs are screened and selected for their ability to inhibit or suppress T cell proliferation according to their T cell proliferation index. In an embodiment, the T cell proliferation index corresponds to the total number of divisions divided by the number of cells that went into division, which takes into account the cells that underwent at least one division, i.e. only responding cells are reflected in the proliferation index. In an embodiment, the hAECs are capable of inhibiting T cell proliferation by a T cell proliferation index of no more than 2.1 or no more than 2.0. In another embodiment, the hAECs are capable of inhibiting T cell proliferation by a T cell proliferation index of no more than 1.9, no more than 1.8, no more than 1.7 or no more than 1.6. In an embodiment, the hAECs are capable of inhibiting T cell proliferation by a T cell proliferation index of up to about 1.7. In an embodiment, the hAECs are capable of inhibiting T cell proliferation by a T cell proliferation index of up to 1.6. In another embodiment, the hAECs are capable of inhibiting T cell proliferation by a T cell proliferation index of no more than 1.5, no more than 1.4, no more than 1.3, no more than 1.2, no more than 1.1 or no more than 1.0. In an embodiment, the hAECs are capable of inhibiting T cell proliferation by a T cell proliferation index of up to about 1.5. In another embodiment, the hAECs are capable of inhibiting T cell proliferation by a T cell proliferation index of up to about 1.4. In an embodiment, the hAECs are capable of inhibiting T cell proliferation by a T cell proliferation index of up to about 1.3. In an embodiment, the hAECs are capable of inhibiting T cell proliferation by a T cell proliferation index of up to about 1.2. In an embodiment, the hAECs are capable of inhibiting T cell proliferation by a T cell proliferation index of up to about 1.1. In an embodiment, the hAECs are capable of inhibiting T cell proliferation by a T cell proliferation index of about 1.

In some embodiments, the hAECs are derived from one or more pre-term placentas. In some embodiments, the hAECs may be derived from one or more term placentas.

In some embodiments, from the population of hAECs proposed for administration to the subject, a sample of hAECs from said population is screened and selected for the cell markers and cell viability. In an embodiment, the sample of hAECs is screened and selected based on their EpCAM, CD90, CD105, CD45 and/or CK-7 expression profiles. In an embodiment, the sample of hAECs is screened and selected for the cell-surface marker EpCAM and the negative for MSC markers, CD90 and CD105. In an embodiment, the sample of hAECs is screened and selected for absence of non-epithelial cells markers CD105 and CD45. In one embodiment, the sample of hAECs is screened and selected for absence of CD45 and for the expression of CK7. In one embodiment, the sample of hAECs is screened and selected for the expression of CK-7. In some embodiments, the sample of hAECs is screened and selected for their ability to suppress T cell activity. In an embodiment, the sample of hAECs is screened and selected for their ability to suppress T cell proliferation.

In some embodiments, the hAECs are screened and selected for their ability to suppress the proliferation of human CD4+ T cell. In some embodiments, the human CD4+ T cell are activated to proliferate with one or more anti-CD3 and CD28 antibodies. In some embodiments, the hAECs are screened and selected for their ability to suppress T cell proliferation when the activated T cells are co-cultured with the hAECs at a ratio of from about 1:1 to about 1:50 (T cells:hAECs). In some embodiments, the hAECs are screened and selected for their ability to suppress T cell proliferation when the activated T cells are co-cultured with the hAECs at a ratio of from about 1:1 to about 1:40. In some embodiments, the hAECs are screened and selected for their ability to suppress T cell proliferation when the activated T cells are co-cultured with the hAECs at a ratio of from about 1:1 to about 1:30. In some embodiments, the hAECs are screened and selected for their ability to suppress T cell proliferation when the activated T cells are co-cultured with the hAECs at a ratio of from about 1:1 to about 1:20. In some embodiments, the hAECs are screened and selected for their ability to suppress T cell proliferation when the activated T cells are co-cultured with the hAECs at a ratio of from about 1:1 to about 1:10. In some embodiments, the hAECs are screened and selected for their ability to suppress T cell proliferation when the activated T cells are co-cultured with the hAECs at a ratio of from about 1:1 to about 1:5. In some embodiments, the hAECs are screened and selected for their ability to suppress T cell proliferation when the activated T cells are co-cultured with the hAECs at a ratio of from about 1:1 to about 1:2. In some embodiments, the hAECs are screened and selected for their ability to suppress T cell proliferation when the activated T cells are co-cultured with the hAECs at a ratio of from about 1:2 to about 1:20. In some embodiments, the hAECs are screened and selected for their ability to suppress T cell proliferation when the activated T cells are co-cultured with the hAECs at a ratio of from about 1:3 to about 1:20. In some embodiments, the hAECs are screened and selected for their ability to suppress T cell proliferation when the activated T cells are co-cultured with the hAECs at a ratio of from about 1:4 to about 1:20. In some embodiments, the hAECs are screened and selected for their ability to suppress T cell proliferation when the activated T cells are co-cultured with the hAECs at a ratio of from about 1:5 to about 1:20. In some embodiments, the hAECs are screened and selected for their ability to suppress T cell proliferation when the activated T cells are co-cultured with the hAECs at a ratio of from about 1:6 to about 1:20. In some embodiments, the hAECs are screened and selected for their ability to suppress T cell proliferation when the activated T cells are co-cultured with the hAECs at a ratio of from about 1:7 to about 1:20. In some embodiments, the hAECs are screened and selected for their ability to suppress T cell proliferation when the activated T cells are co-cultured with the hAECs at a ratio of from about 1:8 to about 1:20. In some embodiments, the hAECs are screened and selected for their ability to suppress T cell proliferation when the activated T cells are co-cultured with the hAECs at a ratio of from about 1:9 to about 1:20. In some embodiments, the hAECs are screened and selected for their ability to suppress T cell proliferation when the activated T cells are co-cultured with the hAECs at a ratio of from about 1:10 to about 1:20. In some embodiments, the hAECs are screened and selected for their ability to suppress T cell proliferation when the activated T cells are co-cultured with the hAECs at a ratio of from about 1:8 to about 1:15. In some embodiments, the hAECs are screened and selected for their ability to suppress T cell proliferation when the activated T cells are co-cultured with the hAECs at a ratio of from about 1:9 to about 1:12. In some embodiments, the hAECs are screened and selected for their ability to suppress T cell proliferation when the activated T cells are co-cultured with the hAECs at a ratio of about 1:10. In some embodiments, the ability of the hAECs to suppress T cell proliferation is determined at from about 2 hours to about 10 days after co-culture. In some embodiments, the ability of the hAECs to suppress T cell proliferation is determined at from about 4 hours to about 9 days of co-culture. In some embodiments, the ability of the hAECs to suppress T cell proliferation is determined at from about 6 hours to about 8 days of co-culture. In some embodiments, the ability of the hAECs to suppress T cell proliferation is determined at from about 8 hours to about 7 days of co-culture. In some embodiments, the ability of the hAECs to suppress T cell proliferation is determined at from about 10 hours to about 6 days of co-culture. In some embodiments, the ability of the hAECs to suppress T cell proliferation is determined at from about 12 hours to about 5 days of co-culture. In some embodiments, the ability of the hAECs to suppress T cell proliferation is determined at from about 24 hours to about 5 days of co-culture. In some embodiments, the ability of the hAECs to suppress T cell proliferation is determined at from about 2 days to about 5 days of co-culture. In some embodiments, the ability of the hAECs to suppress T cell proliferation is determined at from about 3 days to about 5 days of co-culture. In some embodiments, the ability of the hAECs to suppress T cell proliferation is determined at about 4 days of co-culture. In some embodiments, the hAECs are screened and selected for their ability to suppress T cell proliferation by at least about 25% as determined at about 4 days of co-culture.

In some embodiments, the methods described herein may further comprise screening and selecting hAEC for one or more additional criteria. In an embodiment, the one or more additional criteria comprises cell viability, the induction or suppression of specific cytokines and/or the activation, inhibition or proliferation of target cells. In an embodiment, the one or more additional criteria comprises cell viability. Without being bound by theory or by a particular mode of application, it is to be understood that the higher the viability of the hAEC, the greater the therapeutic effect. However, the hAEC may still advantageously be capable of treating IBD in accordance with the methods disclosed herein, even where the viability may otherwise be considered low (e.g., <50%), Where the viability of the hAEC is considered to be low, it may be desirable to increase the number of hAEC administered to the subject. In an embodiment, the hAEC screened and selected for administration to the subject have a viability score of at least about 50%, preferably at least 55%, preferably at least 60%, preferably at least 65%, preferably at least 70%, preferably at least 75%, preferably at least 80%, preferably at least 85%, preferably at least 90%, preferably at least 95%, more preferably at least 99%. In an embodiment, the hAEC screened and selected for administration to the subject have a viability score of from about 70% to about 95%, preferably from about 75% to about 90%.

### 6. Treatment Outcomes

There is provided herein, a method for treating an inflammatory bowel disease in a subject, the method comprising administering to a subject in need thereof a therapeutically effective amount of human amnion epithelial cells (hAECs), wherein prior to administration, the hAECs are screened and selected for their ability to suppress T cell activity. In some embodiments, the inflammatory bowel disease is Crohn's Disease. In some embodiments, the subject has perianal fistulising Crohn's Disease. In some embodiments, the subject has a complex perianal fistula. In a particular embodiment, the subject has a complex perianal fistula that is refractory to treatment with an immune modulating agent for a period of at least about 3 months.

Lack of response may suitably be assessed using routine methods or indices to quantify the symptoms of patient, that would be familiar to persons skilled in the art. Participants were defined as refractory if the external opening of their perianal fistula(s) failed to close without abscess formation despite any of the following: antibiotics for >1 month, immunomodulators or anti-TNF induction or maintenance therapy for >3 months. In an embodiment, the subject was refractory to anti-tumour necrosis factor-α (anti-TNFα) treatment and/or antibiotics. In an embodiment, the subject was refractory to antibiotics (no response after 1 month). In another embodiment the subject was refractory to immunomodulator therapy (no response after 3 months).

In another embodiment, the subject has perianal fistulising Crohn's Disease and said treatment provides a reduction in active fistula number and/or size at 24 weeks following administration of the hAECs. In yet another embodiment, the subject has perianal fistulising Crohn's Disease and said treatment provides a reduction in active fistula number and/or size at 52 weeks following administration of the hAECs. In yet another embodiment, the subject has perianal fistulising Crohn's Disease and said treatment provides a reduction in active fistula number and/or size at 104 weeks following administration of the hAECs.

In an embodiment, the subject has perianal fistulising Crohn's Disease and said treatment provides a reduction in active disease markers and/or disease activity indices. In some embodiments, the treatment provides a reduction in active disease markers and/or disease activity indices at 24 weeks following administration of the hAECs. In some embodiments, the treatment provides a reduction in active disease markers and/or disease activity indices at 52 weeks following administration of the hAECs. In yet another embodiment, the subject has perianal fistulising Crohn's Disease and said treatment provides a reduction in active disease markers and/or disease activity indices at 104 weeks following administration of the hAECs.

In an embodiment, the subject has perianal fistulising Crohn's Disease and said treatment provides a complete response. In some embodiments, the treatment provides a complete response at 24 weeks following administration of the hAECs. In some embodiments, the treatment provides a complete response at 52 weeks following administration of the hAECs. In some embodiments, the treatment provides a complete response at 104 weeks following administration of the hAECs. In another embodiment, the subject has perianal fistulising Crohn's Disease and said treatment provides a partial response. In some embodiments, the treatment provides a partial response at 24 weeks following administration of the hAECs. In some embodiments, the treatment provides a partial response at 52 weeks following administration of the hAECs. In some embodiments, the treatment provides a partial response at 104 weeks following administration of the hAECs.

Methods of determining (measuring) treatment outcomes will be familiar to persons skilled in the art, illustrative examples of which include clinical assessment, blood test (measurement of circulating biomarkers of inflammatory bowel disease, such as C-reactive protein (CRP), erythrocyte sedimentation rate, antineutrophil cytoplasmic antibodies, anti-Saccharomyces cerevisiae antibodies, leucine-rich α2 glycoprotein, and faecal calprotectin (FCP), colonoscopy to assess Crohn's luminal disease activity, and MRI pelvis. A complete response may suitably be defined by the absence of fistula drainage with closure of treated external openings and absence of collection (>2 cm diameter) on MRI. A partial response may be defined by closure of >50% of all treated external openings or >50% reduction of fistula diameter on MRI (if a single fistula), also in the absence of an abscess on MRI. A complete response or partial response can also be assessed using any one of the disease scoring indices or active disease markers, as disclosed herein.

Other suitable methods of assessing treatment outcomes would be familiar to persons skilled in the art, illustrative example of which include determining original and modified Van Assche indices, which are a semiquantitative scoring systems used to assess the severity and disease response, in particular in perianal fistulizing Crohn's Disease. Modified Van Assche index (mVAI) (see, for example, Samaan et al. 2017 Aliment Pharmacol Ther 46:516-28) is a magnetic resonance imaging-based scoring system, which takes into account T1 hyperintensity and infiltrate to the score. A high MRI-based score was confirmed to be associated with more severe clinical behavior. Another validated MRI-based fistula activity assessment score is the MAGNIFI-CD (see, for example, Hindryckx et al. 2019 Gastroenterology 157:1233-44). Another method of measuring severity of disease includes the Perianal disease activity index (PDAI). The perianal disease activity index (PDAI) score includes the evaluation of 5 elements: fistula discharge, pain and restriction of activities, sexual activity restriction, type of perianal disease, and degree of induration (see, for example, Irvine 1995 J Clin Gastroenterol. 20:27-32).

Other methods or indices used to quantify the symptoms of patient with IBD, which can also be used to assess the treatment outcomes, is the Crohn's Disease Activity Index (CDAI) (see, for example, Best et al. 1976 Gastroenterology 70:439-444). The Harvey-Bradshaw index (HBI) was later developed as a simpler version of the CDAI and consists only of clinical parameters, and does not require biochemical tests (see, for example, Harvey and Bradshaw 1980 Lancet 1:514).

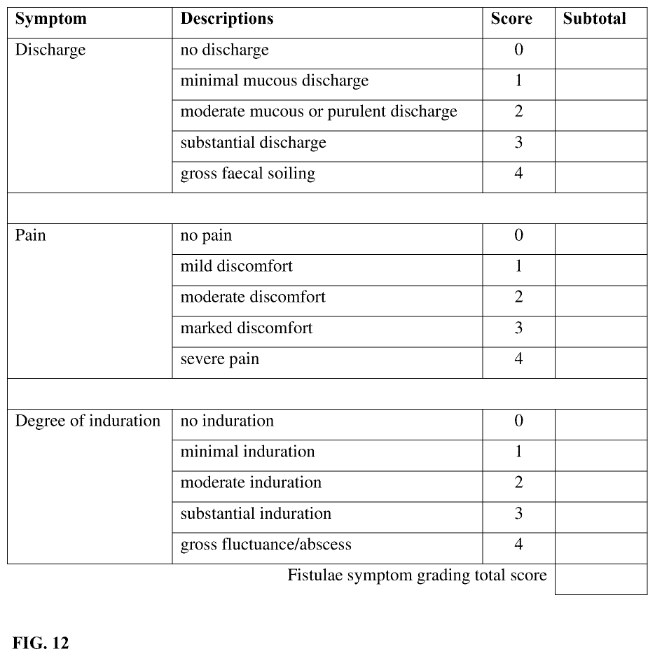

In IBD, health-related quality of life (HRQoL) may also be used to measure illness perception by the patient. The Inflammatory Bowel Disease Questionnaire (IBDQ) is a widely used questionnaire for HRQoL assessment in IBD patients (see, for example, Guyatt et al. 1989 Gastroenterology 96:804-810). A validated, shorter form of this questionnaire (short IBD Questionnaire; sIBDQ) is also widely known and used as a HRQoL measure in IBD. Clinical assessment of fistulas can be scored using the fistula assessment score (FAS), which is a clinical examination-based scoring system used by the Australian Department of Health and Human Services to assess activity of perianal fistulas for qualification for biologic drug use under Australian Pharmaceutical Benefits Scheme (PBS) subsidy. Briefly, and as further described elsewhere herein, the number of externally draining fistulas is recorded, and the fistula symptoms of discharge, pain and degree of induration are scored on a scale of 1-4 (FIG. 12).

Treatment outcomes can be assessed using any one of the methods disclosed herein.

In order that the invention may be readily understood and put into practical effect, particular preferred embodiments will now be described by way of the following non-limiting example.

### EXAMPLES

### Example 1: Isolation of Human Amnion Epithelial Cells

Methods of hAEC isolation for clinical use would be known to the person skilled in the art, for example, the protocols and procedures disclosed in Murphy et al. 2010 Curr Protoc Stem Cell Biol Chapter 1: Unit 1E 6. hAEC were isolated from placentas of consenting healthy mothers screened for opportunistic infections and enriched for cells expressing the cell-surface marker EpCAM and the negative for MSC markers, CD90 and CD105. Sterile hAEC that fulfilled cell viability and T cell activity criteria were cryopreserved and later thawed for immediate injection to the subject.

### Example 2: Screening and Selection of Human Amnion Epithelial Cells

The hAEC were screened and selected for their ability to suppress T cell activity prior to use.

A T-cell proliferation potency assay using human CD4+ cells was used to assay the ability of the hAEC to suppress T cell proliferation.

Briefly, T cells were derived from the peripheral blood from healthy donors and then stained with a viability dye and activated with Dynabeads coated with anti-CD3 and anti-CD28 antibodies. The activated T cells were then mixed with hAECs at a 1:10 ratio (80,000 T cells to 800,000 hAECs per well of a 96-well plate). The T cells were kept in co-culture with the hAECs for 4 days prior to assessment of proliferation by flow cytometry. hAEC that were capable of inhibiting T cell proliferation by at least 25% were selected for subsequent therapeutic use.

### Example 3: Patient Cohort and Treatment Protocol

Ten patients with active complex perianal Crohn's fistulas were selected for the trial. These patients presented with complex perianal fistulas as classified by the American Gastroenterological Association. These patients either had one or more of: high intersphincteric; high trans-sphincteric; extra-sphincteric; supra-sphincteric; two or more external openings or associated collections, and fistulas with a maximum of 2 internal and 3 external openings, that were draining for more than 6 weeks prior to inclusion.

These patients were refractory to anti-tumour necrosis factor-α (anti-TNFα) treatment and/or antibiotics (no response after 1 month) or immunomodulators (no response after 3 months). Definition of lack of response were assessed as per the Perianal Disease Activity Index (PDAI). Participants were defined as refractory if the external opening of their perianal fistula(s) failed to close without abscess formation despite any of the following: antibiotics for >1 month, immunomodulators or anti-TNF induction or maintenance therapy for >3 months.

The hAECs were from 4 donor cell lines. Table 1 shows donor cell line viability at isolation.

TABLE 1
Donor cell viability at isolation

| Cell line | Viability |
|---|---|
| Donor cell line 1 | 87.5% |
| Donor cell line 2 | 74.0% |
| Donor cell line 3 | 90.8% |
| Donor cell line 4 | 84.1% |

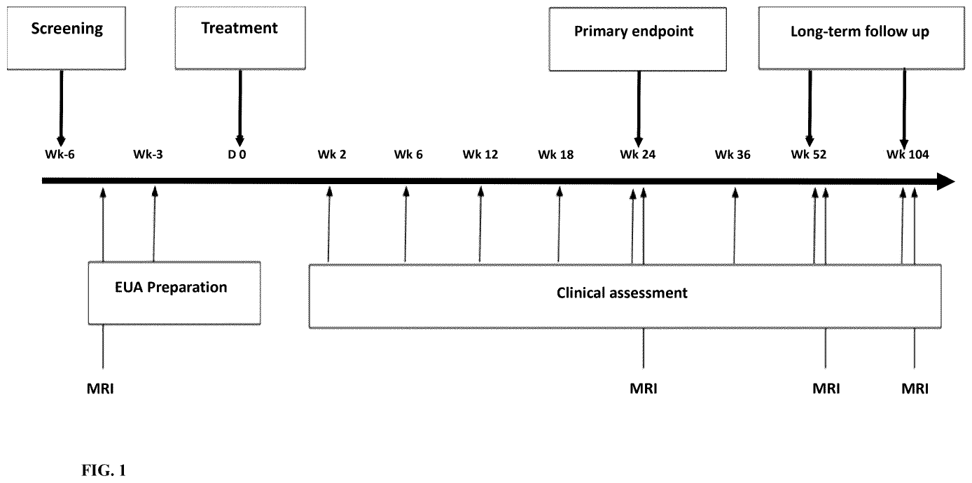

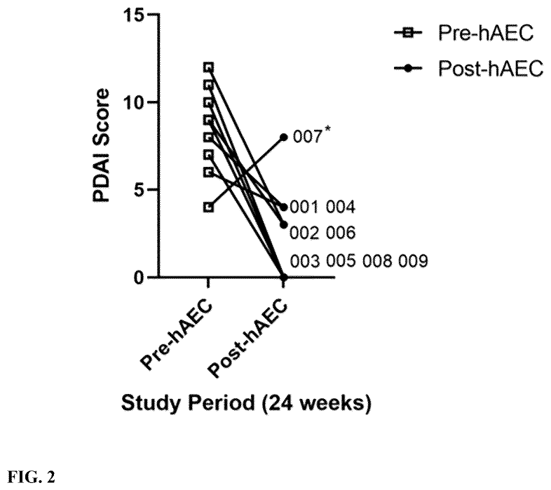

Baseline evaluation included clinical assessment, routine blood tests, colonoscopy to assess Crohn's luminal disease activity and MRI pelvis. A preparatory examination under anaesthesia was performed 2 to 4 weeks before hAEC injection to allow for characterisation of the fistula tract (243 s), debridement and seton placement to adequately drain any collections. Follow up visits included clinical assessments and routine blood tests with a clinician and a colorectal surgeon up until week 104. The study outline is shown in FIG. 1.

The patients received a single local dose of 40 million hAEC per fistula with concurrent closure of the fistula internal orifice. Up to 3 fistulas were treated per participant (maximum 120 million cells). Patients continued maintenance biologic and immunomodulator therapies. Study outcomes were assessed at week 24 after hAEC injection with follow up for at least 52 weeks, and preferably 104 weeks. Table 2 provides the result of the analysis of hAEC viability during infusion.

TABLE 2
Cell viability during hAEC infusion

| | | Viability | Viability |
|---|---|---|---|
| Patient | hAEC Cell line | at infusion | post-infusion |
| 001 | Donor cell line 1 | 81.7% | 80.20% |
| 002 | Donor cell line 1 | | 87.70% |
| 003 | Donor cell line 1 | 78.9% | 74.90% |
| 004 | Donor cell line 4 | | 80.20% |
| 005 | Donor cell line 4 | 85.9% | 88.50% |
| 006 | Donor cell line 3 | 79.8% | 79.20% |
| 007 | Donor cell line 2 | | 79.20% |
| 008 | Donor cell line 2 | 87.7% | 79.30% |
| 009 | Donor cell line 3 | 89.5% | 90.10% |
| 010 | Donor cell line 4 | 80.5% | 74.40% |

### Example 4: Human Amnion Epithelial Cells for the Treatment for Refractory Complex Fistulizing Perianal Crohn's Disease

After 52 weeks, the follow up data indicates there have been no suspected unexpected serious adverse reactions. The most common adverse event was post-operative anal pain requiring oral analgesia.

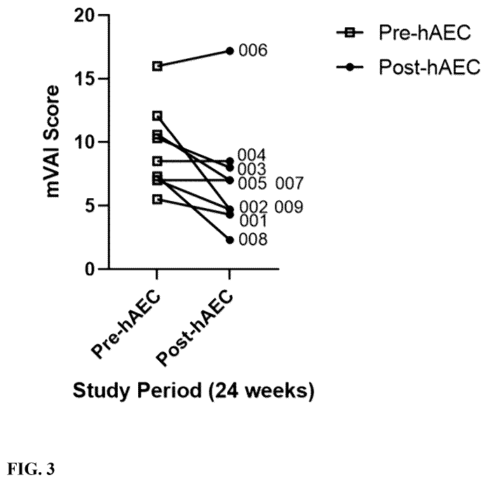

At 24 weeks, data from 9 patients were available. Of the 9 patients, 3 completely responded (closure of all external fistula orifices without abscess on MRI) and 4 partially responded (≥50% reduction in active fistula number or size). Only 1 was a non-responder but still reported a reduction in the Perianal Disease Activity Index (PDAI). Paired t-tests assessing baseline and week 24 outcomes showed a mean reduction of 6 points for the PDAI (95% CI −9.6 to −2.4, p=0.005) (FIG. 2); a mean reduction of 2.3 points for the modified Van Assche MRI index (95% CI −4.34 to −0.20, p=0.035) (FIG. 3). The study protocol was successful and well-tolerated. There was no statistical difference between baseline and week 24 quality of life (p=0.223) measured by the short IBD Questionnaire.

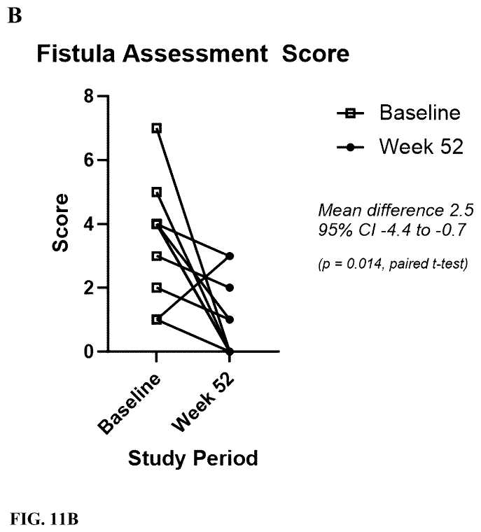

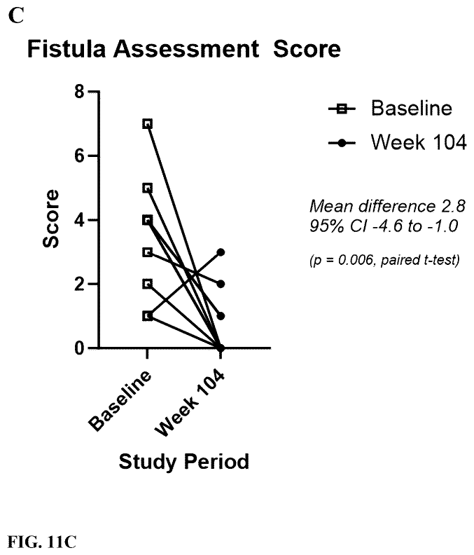

At week 2, there was a mean difference in HBI-2 points (95% CI −5 to 1, p=0.208) and a mean reduction of 66 points for the Crohn's Disease Activity Index (95% CI −122 to −11, p=0.025) (FIG. 11).

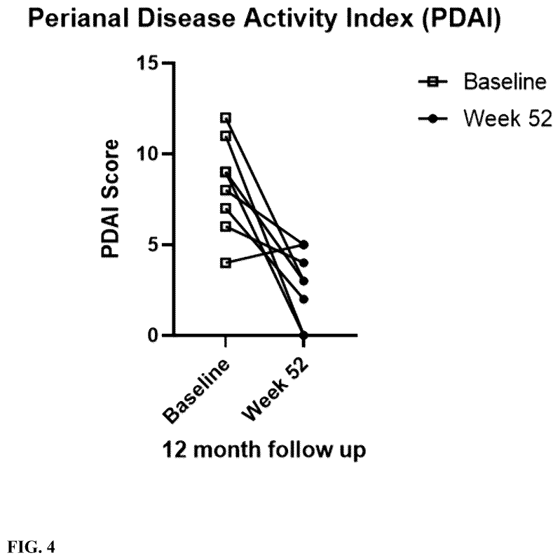

8 of the 10 patients have been assess for the 52 week timepoint using the PDAI. Paired t-tests assessing baseline and week 52 outcomes showed a mean reduction of 5.5 points for the PDAI (95% CI −8.9 to −2.1, p=0.007) (FIG. 4).

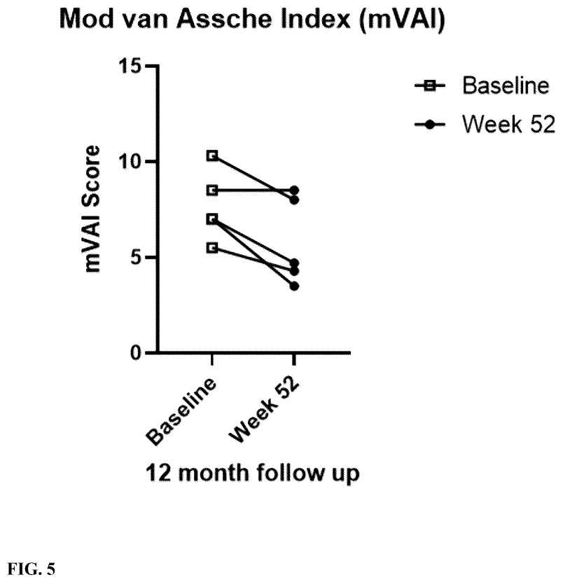

5 of the 10 patients have been assess for the 52 week timepoint using the modified van Assche MRI index. Paired t-tests assessing baseline and week 52 outcomes showed a mean reduction of 1.9 points for the PDAI (95% CI −3.5 to −0.22, p=0.035) (FIG. 5).

### Example 5: Summary of Data from 10 Patients

At week 24, 8 fistulas out of the total 15 (53%) belonging to the ten patients, achieved closure of the external opening without any abscess. Complete response was achieved in 4 patients and a partial response occurred in a further 4 patients. The remaining two patients who did not fit the study definition of treatment response at week 24 nevertheless demonstrated some improvement, insofar as (i) one participant reported a reduced PDAI score from 7 to 2 with reduced fistula enhancement on MRI compared with baseline, but pin-point non-draining external fistula openings persisted and (ii) the other participant demonstrated a reduction in PDAI score from 8 to 4 with stable fistula activity on MRI.

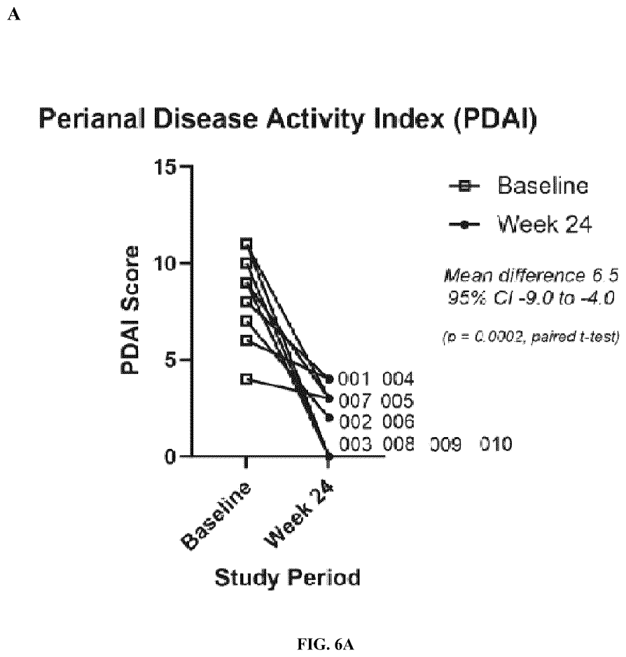

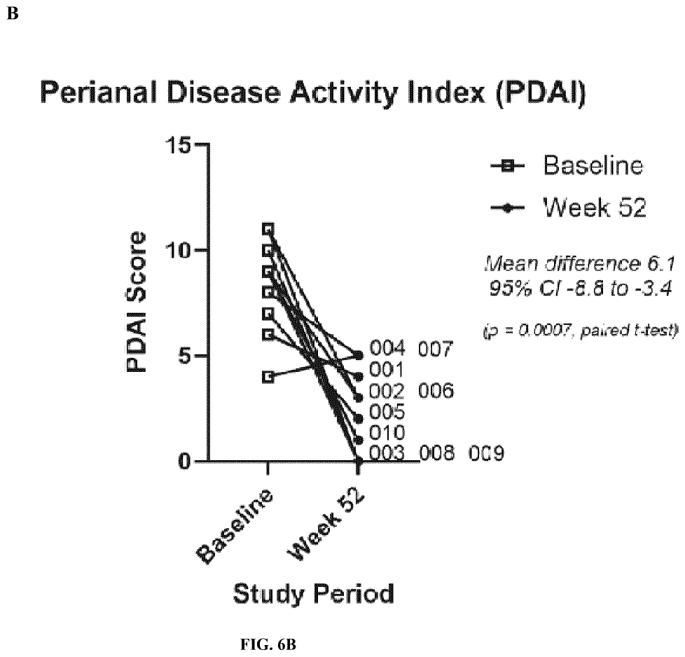

Overall, there was a mean reduction in week 24 PDAI scores compared with baseline of 6.5 points (95% CI −9.0 to −4.0, p=0.0002, paired t-test) as shown in FIG. 6A. All participants at week 24 reported a PDAI score of <4 (accepted as inactive) and 8 remained seton free at that timepoint (setons were re-inserted in two participants at week 23).

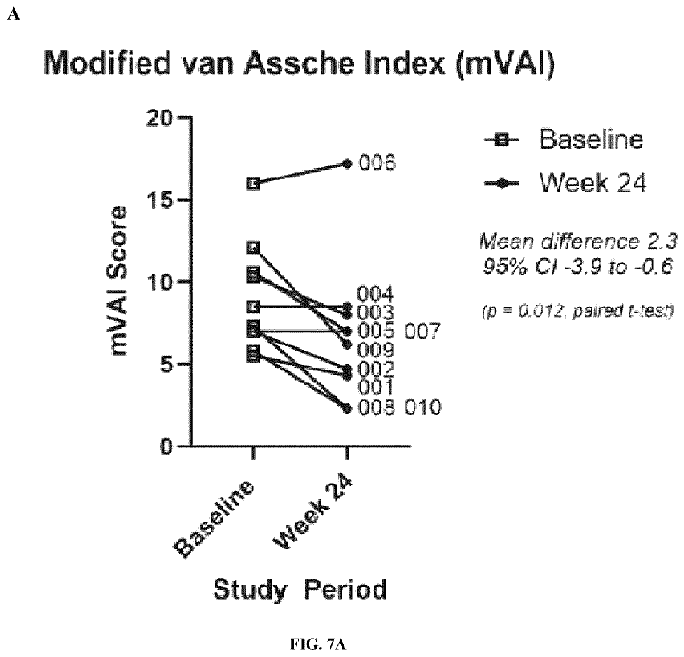

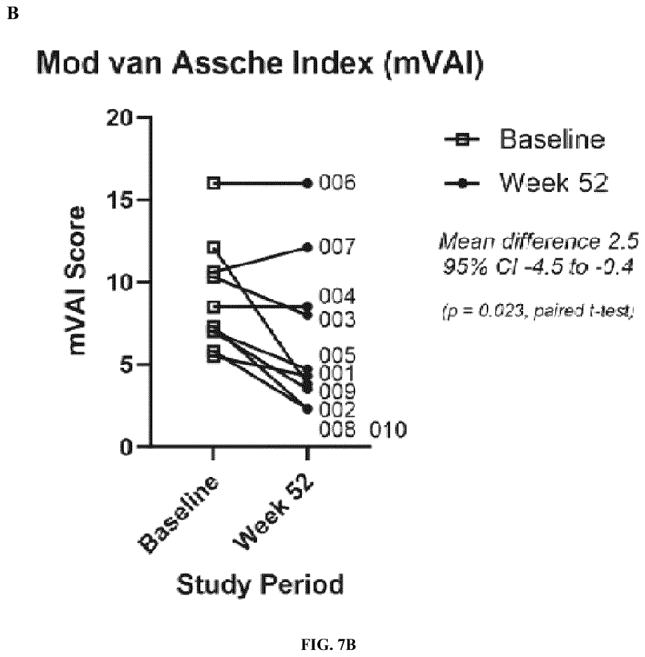

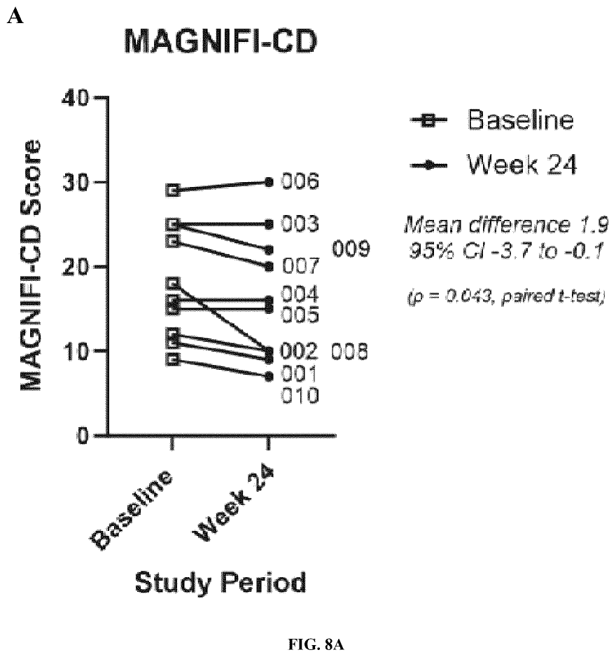

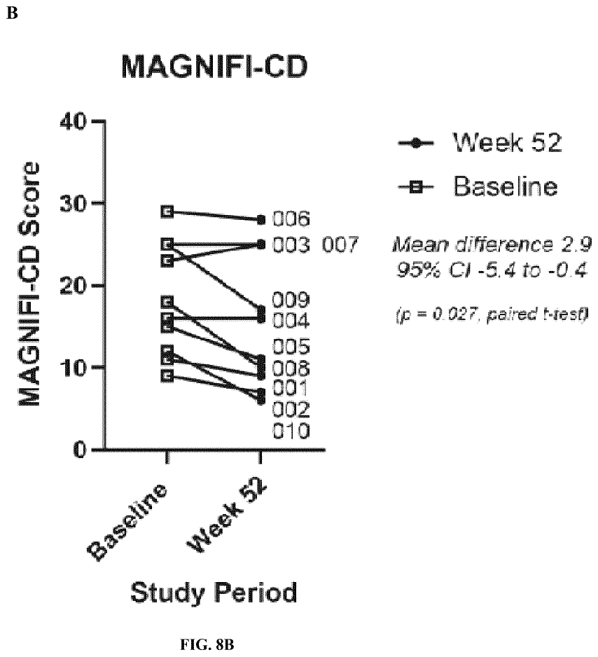

There was a mean reduction in mVAI (FIG. 7A) and MAGNIFI-CD (FIG. 8A) scores at week 24 by a mean of 2.3 points (95% CI −3.9 to −0.6, p=0.012, paired t-test) and 1.9 points (95% CI −3.7 to −0.1, p=0.043, paired t-test), respectively.

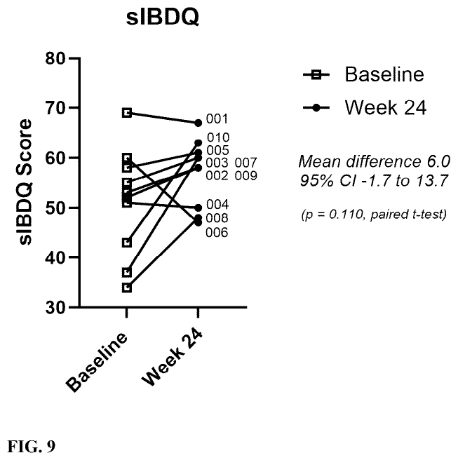

There was a mean difference of 6.0 points (95% CI −1.7 to 13.7, p=0. 110, paired t-test) in the sIBDQ scores at baseline and week 24 overall and 7 of the participants reported an improvement in sIBDQ scores. Participants who were complete responders (4 patients) reported a higher improvement in sIBDQ scores than the 6 patients who were non-complete responders by a mean difference of 15.8 points (95% CI 4.9 to 26.8, p=0.01, paired t-test). FIG. 9 shows baseline and week 24 sIBDQ scores.

At week 52 after hAEC injection, there were no suspected unexpected serious adverse reactions. All fistulas with closed external openings (n=8) remained closed without any abscess at week 52. All complete responders remained in clinical remission with closure of the external fistula opening, absence of significant symptoms and absence of abscess on serial MRI pelvis.

The persistence of response assessed by the PDAI score was maintained at week 52 with a mean reduction of 6.1 points (95% CI −8.8 to −3-4, p=0.0007, paired t-test) compared with baseline (FIG. 6B). The MRI fistula activity indices also maintained their improvement at week 52 compared with baseline with the mVAI (FIG. 7B) demonstrating a mean reduction of 2.5 points (95% CI −4.5 to −0.4, p=0.023, paired t-test) and the MAGNIFI-CD (FIG. 8B) demonstrating a mean reduction of 2.9 points (95% CI −5.4 to −0.4, p=0.027, paired t-test).

### Example 6: Sustained Effect of hAECs for the Treatment for Refractory Complex Fistulizing Perianal Crohn's Disease

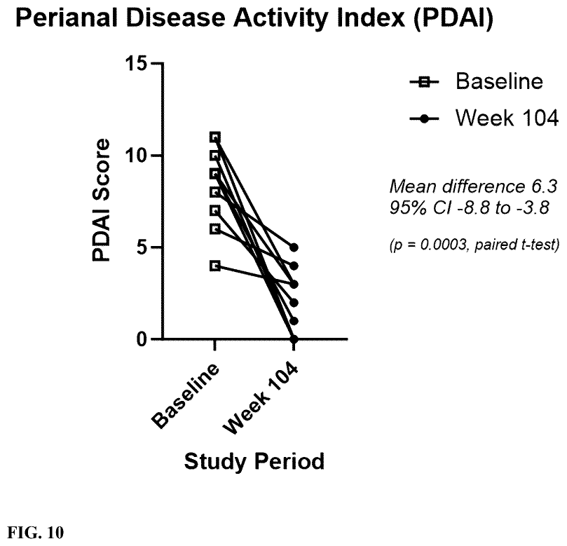

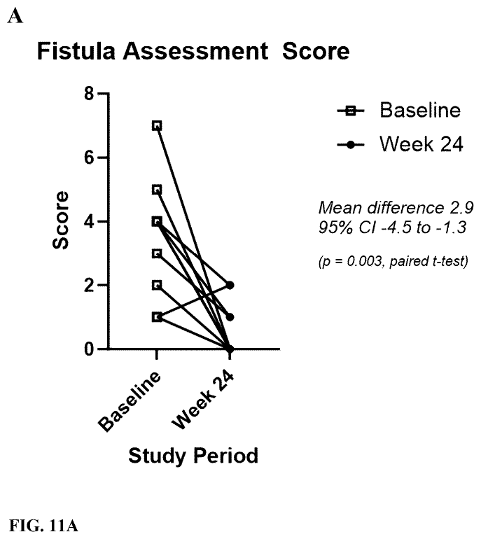

At week 104 of treatment, data from 10 patients indicated a mean reduction of 6.30 points in PDAI comparing baseline to week 104 (95% CI −8.78 to −3.82, p=0.0003) (FIG. 10).

### Example 7: Effect of hAECs on Perianal Fistulas

The Fistula Assessment Score (FAS) was used to assess the activity of perianal fistulas based on clinical examination.

At week 24, there was a mean reduction of 2.9 points for the Fistula Assessment Score (FAS) (95% CI −4.5 to −1.3, p=0.003) (FIG. 11A). At week 52, there was a mean reduction of FAS by 2.50 points (95% CI −4.36 to −0.65, p=0.014) (FIG. 11B). At week 104, there was a mean reduction of FAS by 2.80 points (95% CI −4.58 to −1.02, p=0.006) (FIG. 11C).

These data suggest that local injection of hAEC therapy for refractory complex perianal fistulising Crohn's disease is safe, well-tolerated, feasible and demonstrated improvement. Quality of life was improved, in particular in those patients who achieved complete fistula healing.

### Example 8: Assessment of T Cell Suppression by Donor hAECs

hAECs were isolated from the placentae donated by women with healthy pregnancies (Monash Health, HREC 12223B) as shown in Table 3. hAECs were isolated in compliance with current Good Manufacturing Practices (cGMP). The donor hAECs were assessed for critical quality attributes defined as absence of non-epithelial cells markers CD105, CD45 or CD45 and post thaw viability of >70% with expression of CK-7. For T cell proliferation assay, hAECs were co-cultured with T cells at a ratio of 10:1.

Proliferation capacity of T cells was measured using flow cytometry. Viability was measured by Trypan blue dye exclusion.

TABLE 3
Characteristics of healthy women donors
undergoing caesarian section (n = 31)

| Variable | Mean ± SD |
|---|---|
| Age (Years) | 34.6 ± 4.6 |
| Gestation Weeks | 38.7 ± 0.6 |
| Smoker | |
| Yes | 0 (0%) |
| No | 31 (100%) |

T cell proliferation was evaluated using CellTrace Violet, to track cell proliferation. The T cells were kept in co-culture with the hAECs for 4 days prior to assessment of proliferation by flow cytometry analysis (FlowJo).

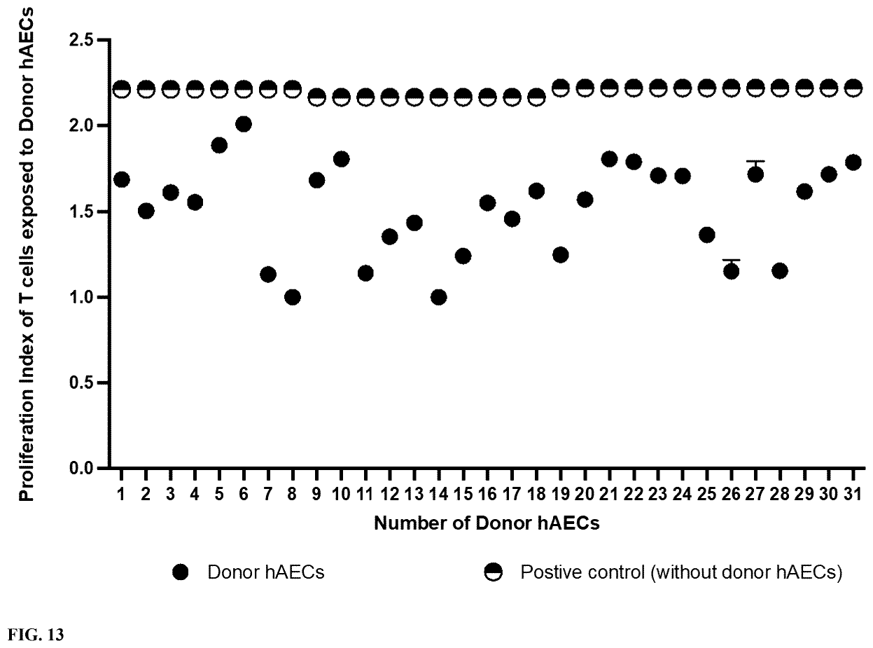

The potency of the hAECs isolated from the 31 placentae at suppressing T cell proliferation is shown in FIG. 13. While the all hAECs showed ability to suppress T cell proliferation, the immunomodulatory potential was different between the different hAECs; from high suppressive T cell proliferation index (e.g. proliferative index of 1) to less suppressive T cell proliferation index (e.g. proliferative index of 2). T cell proliferation index is calculated by taking the total number of divisions divided by the number of cells that went into division. (i.e. the proliferation index only takes into account the cells that underwent at least one division).

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A method for treating a fistula in a subject, the method comprising administering to a subject in need thereof a therapeutically effective amount of human amnion epithelial cells (hAECs), wherein prior to administration, the hAECs are screened and selected for their ability to suppress T cell activity.

2. A method for treating an inflammatory bowel disease in a subject, the method comprising administering to a subject in need thereof a therapeutically effective amount of human amnion epithelial cells (hAECs), wherein prior to administration, the hAECs are screened and selected for their ability to suppress T cell activity.

3. The method of claim 2, wherein the inflammatory bowel disease is ulcerative colitis.

4. The method of claim 2, wherein the inflammatory bowel disease is Crohn's Disease.

5. The method of claim 2 or claim 4, wherein the inflammatory bowel disease is perianal fistulising Crohn's Disease.

6. The method of any one of claims 1 to 5, wherein the subject has a complex perianal fistula.

7. The method of any one of claims 1 to 6, wherein the hAECs are screened and selected for their ability to suppress T cell proliferation by at least about 25%.

8. The method of any one of claims 1 to 7, wherein the subject has one or more of intersphincteric, trans-sphincteric; extra-sphincteric; and supra-sphincteric fistulas.

9. The method of any one of claims 1 to 8, wherein the subject has a rectovaginal fistula.

10. The method of any one of claims 5 to 9, wherein the subject has a complex perianal fistula that is refractory to treatment with an immune modulating agent, such as an anti-tumour necrosis factor-α (TNF-α) antibody for a period of at least about 3 months.

11. The method of any one of claims 1 to 10, wherein the hAEC are administered to a fistula of the subject in an amount of from about 1×10⁶ to about 1×10⁷ hAECs per fistula.

12. The method of any one of claims 1 to 10, wherein the hAEC are administered to a fistula of the subject in an amount of from about 10×10⁶ to about 80×10⁶ hAECs per fistula.

13. The method of any one of claims 1 to 10, wherein the hAEC are administered to a fistula of the subject in an amount of from about 30×10⁶ to about 50×10⁶ hAECs per fistula.

14. The method of any one of claims 1 to 10, wherein the hAEC are administered to a fistula of the subject in an amount of about 40×10⁶ hAECs per fistula.

15. The method of any one of claims 1 to 14, wherein the subject is a human.

16. The method of any one of claims 1 to 15, wherein the hAECs are autologous or allogenic or xenogeneic to the subject.

17. The method of claim 16, wherein the hAECs are allogeneic to the subject.

18. The method of any one of claims 1 to 17, wherein the hAECs are immortalised hAECs.

19. The method of any one of claims 1 to 18, wherein following said screening and selection, the hAECs are cryopreserved and subsequently thawed immediately prior to administration to the subject.

20. The method of any one of claims 1 to 19, wherein said treatment provides a reduction in active fistula number and/or size at 24 weeks following administration of the hAECs.

21. The method of any one of claims 1 to 20, wherein said treatment provides a reduction in active fistula number and/or size at 52 weeks following administration of the hAECs.

22. The method of any one of claims 1 to 21, wherein said treatment provides a reduction in active fistula number and/or size at 104 weeks following administration of the hAECs.

22. Use of human amnion epithelial cells (hAECs) in the manufacture of a medicament for treating a fistula in a subject, wherein the hAECs formulated for administration to a subject in need thereof, and wherein prior to said administration, the hAECs are screened and selected for their ability to suppress T cell activity

24. Use of human amnion epithelial cells (hAECs) in the manufacture of a medicament for treating inflammatory bowel disease in a subject, wherein the hAECs formulated for administration to a subject in need thereof, and wherein prior to said administration, the hAECs are screened and selected for their ability to suppress T cell activity.

25. The use of claim 24, wherein the inflammatory bowel disease is ulcerative colitis.

26. The use of claim 24, wherein the inflammatory bowel disease is Crohn's Disease.

27. The use of claim 26, wherein the inflammatory bowel disease is perianal fistulising Crohn's Disease.

28. The use of any one of claims 23 to 27, wherein the subject has a complex perianal fistula.

29. The use of any one of claims 23 to 28, wherein the hAECs are screened and selected for their ability to suppress T cell proliferation by at least about 25%.

30. The use of any one of claims 23 to 29, wherein the subject has one or more of intersphincteric, trans-sphincteric; extra-sphincteric; and supra-sphincteric fistulas.

31. The use of any one of claims 23 to 30, wherein the subject has a rectovaginal fistula.

32. The use of any one of claims 26 to 31, wherein the subject has a complex perianal fistula that is refractory to treatment with an immune modulating agent, such as an anti-tumour necrosis factor-α (TNF-α) antibody, for a period of at least about 3 months.

33. The use of any one of claims 23 to 32, wherein the hAEC are formulated for administration to a fistula of the subject in an amount of from about 1×10⁶ to about 1×10⁷ hAECs per fistula.

34. The use of any one of claims 23 to 33, wherein the hAEC are formulated for administration to a fistula of the subject in an amount of from about 10×10⁶ to about 80×10⁶ hAECs per fistula.

35. The use of any one of claims 23 to 34, wherein the hAEC are formulated for administration to a fistula of the subject in an amount of from about 30×10⁶ to about 50×10⁶ hAECs per fistula.

36. The use of any one of claims 23 to 35, wherein the hAEC are formulated for administration to a fistula of the subject in an amount of about 40×106 hAECs per fistula.

37. The use of any one of claims 23 to 36, wherein the subject is a human.

38. The use of any one of claims 23 to 37, wherein the hAECs are autologous or allogenic or xenogeneic to the subject.

39. The use of claim 38, wherein the hAECs are allogeneic to the subject.

40. The use of any one of claims 23 to 39, wherein the hAECs are immortalised hAECs.

41. The use of any one of claims 23 to 40, wherein following said screening and selection, the hAECs are cryopreserved and subsequently thawed immediately prior to being administered to the subject.

42. The use of any one of claims 23 to 41, wherein said treatment provides a reduction in active fistula number and/or size at 24 weeks following administration of the hAECs.

43. The use of any one of claims 23 to 42, wherein said treatment provides a reduction in active fistula number and/or size at 52 weeks following administration of the hAECs.

44. Human amnion epithelial cells (hAECs) for use in the treatment of a fistula in a subject, wherein said treatment comprises administering to a subject in need thereof a therapeutically effective amount of the hAECs, wherein prior to administration to the subject, the hAECs are screened and selected for their ability to suppress T cell activity.

45. Human amnion epithelial cells (hAECs) for use in the treatment of inflammatory bowel disease in a subject, wherein said treatment comprises administering to a subject in need thereof a therapeutically effective amount of the hAECs, wherein prior to administration to the subject, the hAECs are screened and selected for their ability to suppress T cell activity.

46. The hAECs of claim 45, wherein the inflammatory bowel disease is ulcerative colitis.

47. The hAECs of claim 45, wherein the inflammatory bowel disease is Crohn's Disease.

48. The hAECs of claim 47, wherein the inflammatory bowel disease is perianal fistulising Crohn's Disease.

49. The hAECs of any one of claims 44 to 48, wherein the subject has a complex perianal fistula.

50. The hAECs of any one of claims 44 to 49, wherein the hAECs are screened and selected for their ability to suppress T cell proliferation by at least about 25%.

51. The hAECs of any one of claims 44 to 50, wherein the subject has one or more of intersphincteric, trans-sphincteric; extra-sphincteric; and supra-sphincteric fistulas.

52. The hAECs of any one of claims 44 to 51, wherein the subject has a rectovaginal fistula.

53. The hAECs of any one of claims 49 to 52, wherein the subject has a complex perianal fistula that is refractory to treatment with an immune modulating agent, such as an anti-tumour necrosis factor-α (TNF-α) antibody, for a period of at least about 3 months.

54. The hAECs of any one of claims 44 to 53, wherein the hAEC are administered to a fistula of the subject in an amount of from about 1×10⁶ to about 1×10⁷ hAECs per fistula.

55. The hAECs of any one of claims 44 to 54, wherein the hAEC are administered to a fistula of the subject in an amount of from about 10×10⁶ to about 80×10⁶ hAECs per fistula.

56. The hAECs of any one of claims 44 to 55, wherein the hAEC are administered to a fistula of the subject in an amount of from about 30×10⁶ to about 50×10⁶ hAECs per fistula.

57. The hAECs of any one of claims 44 to 56, wherein the hAEC are administered to a fistula of the subject in an amount of about 40×10⁶ hAECs per fistula.

58. The hAECs of any one of claims 44 to 57, wherein the subject is a human.

59. The hAECs of any one of claims 44 to 58, wherein the hAECs are autologous or allogenic or xenogeneic to the subject.

60. The hAECs of claim 59, wherein the hAECs are allogeneic to the subject.

61. The hAECs of any one of claims 44 to 60, wherein the hAECs are immortalised hAECs.

62. The hAECs of any one of claims 44 to 61, wherein following said screening and selection, the hAECs are cryopreserved and subsequently thawed immediately prior to administration to the subject.

63. The hAECs of any one of claims 44 to 62, wherein said treatment provides a reduction in active fistula number and/or size at 24 weeks following administration of the hAECs.

64. The hAECs of any one of claims 44 to 63, wherein said treatment provides a reduction in active fistula number and/or size at 52 weeks following administration of the hAECs.

65. The hAECs of any one of claims 44 to 64, wherein said treatment provides a reduction in active fistula number and/or size at 104 weeks following administration of the hAECs.
